(19) United States
(12) Patent Application Publication
(10) Pub. No.: US 2002/0132786 A1
(43) Pub. Date: Sep. 19, 2002

(54) IAP binding peptide or polypeptide and methods of using the same

(76) Inventor: Emad S. Alnemri, (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/939,293

(22) Filed: Aug. 24, 2001

Related U.S. Application Data
(60) Provisional application No. 60/227,735, filed on Aug. 24, 2000.

(51) Int. Cl. A61K 48/00; A61K 38/17; C07H 21/04; C12P 21/02; C12N 5/06; C12N 9/00

(52) U.S. Cl. 514/44; 514/12; 536/23.2; 435/183; 435/69.1; 435/325; 435/320.1

(57) ABSTRACT

An isolated nucleic acid molecule comprising a polynucleotide having a sequence encoding a peptide or polypeptide of Smac having at least two contiguous amino acid residues derived from at least residues 56-139 of SEQ ID NO:1 and of which up to 184 contiguous amino acid residues can be derived from residues 56-239 of SEQ ID NO:1, a functional variant of each or a functional equivalent of each, each of which is capable of specifically binding to at least a portion of an Inhibitor of Apoptosis protein. This peptide can be used in a method to modulate apoptosis or to identify modulators of apoptosis as well as in therapeutic uses.

### CROSS-REFERENCE TO RELATED APPLICATION

This application claims the benefit of U.S. Provisional Patent Application No. 60/227,735, filed Aug. 24, 2000, which is incorporated herein by reference in its entirety.

### BACKGROUND OF THE INVENTION

1. Field of the Invention

The present invention relates generally to the regulation of apoptosis, and more particularly, to Smac and to IAP binding Smac derived and related polypeptides and peptides, and methods of using such polypeptides and peptides to modulate and to identify modulators of apoptosis as well as in therapeutic uses.

2. Description of the Related Art

Apoptosis is a highly conserved cell suicide program essential for development and tissue homeostasis of all metazoan organisms. Changes to the apoptotic pathway that prevent or delay normal cell turnover can be just as important in the pathogenesis of diseases as are abnormalities in the regulation of the cell cycle. Like cell division, which is controlled through complex interactions between cell cycle regulatory proteins, apoptosis is similarly regulated under normal circumstances by the interaction of gene products that either prevent or induce cell death.

Since apoptosis functions in maintaining tissue homeostasis in a range of physiological processes such as embryonic development, immune cell regulation and normal cellular turnover, the dysfunction or loss of regulated apoptosis can lead to a variety of pathological disease states. For example, the loss of apoptosis can lead to the pathological accumulation of self-reactive lymphocytes that occurs with many autoimmune diseases. Inappropriate loss or inhibition of apoptosis can also lead to the accumulation of virally infected cells and of hyperproliferative cells such as neoplastic or tumor cells. Similarly, the inappropriate activation of apoptosis can also contribute to a variety of pathological disease states including, for example, acquired immunodeficiency syndrome (AIDS), neurodegenerative diseases and ischemic injury. Treatments that are specifically designed to modulate the apoptotic pathways in these and other pathological conditions can alter the natural progression of many of these diseases.

Although apoptosis is mediated by diverse signals and complex interactions of cellular gene products, the results of these interactions ultimately feed into a cell death pathway that is evolutionarily conserved between humans and invertebrates. The pathway, itself, is a cascade of proteolytic events analogous to that of the blood coagulation cascade.

Several gene families and products that modulate the apoptotic process have now been identified. Key to the apoptotic program is a family of cysteine proteases termed caspases. The human caspase family includes Ced-3, human ICE (interleukin-1-βconverting enzyme) (caspase-1), ICH-1 (caspase-2), CPP32 (caspase-3), ICEᵣₑₗII (caspase-4), ICEᵣₑₗIII (caspase-5), Mch2 (caspase-6), ICE-LAP3 (casepase-7), Mch5 (caspase-8), ICE-LAP6 (caspase-9), Mch4 (caspase-10), caspase 11-14 and others.

The caspase proteins share several common features. They are cysteine proteases (named for a cysteine residue in the active site) that cleave their substrates after specific aspartic acid residues (Asp-X). Furthermore, caspases are primarily produced as inactive zymogens, known as procaspases, which require proteolytic cleavage at specific internal aspartate residues for activation. The primary gene product is arranged such that the N-terminal peptide (prodomain) precedes a large subunit domain, which precedes a small subunit domain. The large subunit contains the conserved active site pentapeptide QACXG (X=R, Q, G) (SEQ ID NO:3) which contains the nucleophilic cysteine residue. The small subunit contains residues that bind the Asp carboxylate side chain and others that determine substrate specificity. Cleavage of a caspase yields the two subunits, the large (generally approximately 20 kD) and the small (generally approximately 10 kD) subunit that associate non-covalently: to form a heterodimer, and, in some caspases, an N-terminal peptide of varying length. The heterodimer may combine non-covalently to form a tetramer.

Caspase zymogens are themselves substrates for caspases. Inspection of the interdomain linkages in each zymogen reveals target sites (i.e. protease sites) that indicate a hierarchical relationship of caspase activation. By analyzing such pathways, it has been demonstrated that caspases are required for apoptosis to occur. Moreover, caspases appear to be necessary for the accurate and limited proteolytic events that are the hallmark of classic apoptosis (see Salvesen and Dixit, Cell 91:443-446, 1997). During apoptosis, the initiator caspase zymogens are activated by autocatalytic cleavage, which then activate the effector caspases by cleaving their inactive zymogens (Salvesen and Dixit, Proc. Natl. Acad. Sci. USA 96:10964-10967, 1999; Srinivasula et al., Mol. Cell. 1:949-957, 1998). This characteristic indicates that caspases implicated in apoptosis may execute the apoptotic program through a cascade of sequential activation of initiators and effector procaspases (Salvesen and Dixit, Cell 91:443-446, 1997). The initiators are responsible for processing and activation of the effectors. The effectors are responsible for proteolytic cleavage of a number of cellular proteins leading to the characteristic morphological changes and DNA fragmentation that are often associated with apoptosis (reviewed in Cohen, Biochem. J. 326:1-16, 1997; Henkart, Immunity 4:195-201, 1996; Martin and Green, Cell 82:349-352, 1995; Nicholson and Thomberry, TIBS 257:299-306, 1997; Porter et al., BioEssays 19:501-507, 1997; Salvesen and Dixit, Cell 91:443-446, 1997). The first evidence for an apoptotic caspase cascade was obtained from studies on death receptor signaling (reviewed in Fraser and Evan, Cell 85:781-784, 1996; Nagata, Cell 88:355-365, 1997) which indicated that the death signal is transmitted in part by sequential activation of the initiator procaspase-8 and the effector procaspase-3 (Boldin et al., Cell 85:803-815, 1996; Fernandes-Alnemri et al., Proc. Natl. Acad. Sci. USA 93:7464-7469, 1996; Muzio et al., Cell 85:817-827, 1996; Srinivasula et al., Proc. Natl. Acad. Sci. USA 93:13706-13711, 1996). More direct evidence was provided when it was demonstrated that the cytochrome c death signal is transmitted through activation of a cascade involving procaspase-9 and caspase-3 (Li et al., Cell 91:479-489, 1997).

The initiator caspase zymogens are activated by adaptor proteins such as FADD and Apaf-1, which associate in a stimulus-dependent manner with the prodomains of these zymogens and promote their activation via oligomerization (Salvesen and Dixit, Proc. Natl. Acad. Sci. USA 96:10964-10967, 1999; Srinivasula et al., Mol. Cell. 1:949-957, 1998). For example, ligands binding to the cell surface death receptors triggers binding of procaspase-8 to FADD and its subsequent activation and release from the death receptor complex. Likewise, release of cytochrome c from the mitochondria in response to apoptotic stimuli such as serum starvation, ionization radiation, DNA damaging agents etc. triggers oligomerization of Apaf-1 in an ATP or dATP dependent manner. The oligomeric Apaf-1 apoptosome then recruits and activates procaspase-9.

Given the potentially irreversible caspase cascade triggered by activation of the upstream initiator caspases, it is crucial that activation of caspases in the cell be tightly regulated. A number of cellular proteins have been shown to modulate caspase activation and activity. One of these, FLAME/FLIP, inhibits death receptor-mediated activation of caspase-8 by binding to FADD (Irmler et al., Nature 388:190-195, 1997; Srinivasula et al., J. Biol. Chem. 272:18542-18545, 1997). Others, such as the anti-apoptotic members of the Bcl-2 family, inhibit Apaf-1-mediated activation of caspase-9 by blocking cytochrome c release from the mitochondria (reviewed in Adams and Cory, Science 281:1322-1326, 1998; Green and Reed, Science 281:1309-1312, 1998). Heat shock proteins, Hsp70 and Hsp90, also interfere with the mitochondrial apoptotic pathway by modulating the formation of a functional Apaf-1 apoptosome (Saleh, et al., Nature Cell. Biol. 2:476-483, 2000: Pandey, et al., EMBO J. 19:4310-4322, 2000). Finally, members of the Inhibitor of Apoptosis Protein (IAP) family, such as XIAP, c-IAP-1, and c-IAP-2, block both the death receptor and mitochondrial pathways by inhibiting the activity of the effector caspase-3 and caspase-7 and the initiator caspase-9 (reviewed in Deveraux and Reed, Genes Dev. 13:239-252, 1999).

Smac/DIABLO, a mitochondrial protein, which is released together with cytochrome c from the mitochondria in response to apoptotic stimuli, was found to promote caspase activation by binding and neutralizing the IAPs (Du et al., Cell 102:33-42, 2000; Verhagen et al., Cell 102:43-53, 2000).

### SUMMARY OF THE INVENTION

The present invention generally provides nucleic acid molecules the encode peptides or polypeptides of Smac, functional variants of each and functional equivalents of each, peptides or polypeptides of Smac, functional variants of each and functional equivalents of each and methods of using such peptides to modulate and to identify modulators of apoptosis. However, the present invention does not include full length Smac (Smac-L) or nucleic acid molecules encoding same within the scope of the invention. In one aspect, the present invention provides an isolated nucleic acid molecule comprising, consisting essentially of or consisting of a polynucleotide sequence encoding a peptide or polypeptide of Smac having at least two contiguous amino acid residues derived from at least residues 56-139 of SEQ ID NO:1 and of which up to 184 contiguous amino acid residues can be derived from residues 56-239 of SEQ ID NO:1, a functional variant of each or a functional equivalent of each, each of which is capable of specifically binding to at least a portion of an Inhibitor of Apoptosis protein (IAP). In certain embodiments, the portion of the IAP bound is at least one of the BIR domains of IAP, e.g. BIR1, BIR2 and BIR3, or it can be a full length IAP. In one embodiment, the encoded peptide or polypeptide has an amino acid sequence of at least the amino acids Ala-Val. In another embodiment, the encoded peptide or polypeptide has an amino acid sequence provided in SEQ ID NO:13.

In another aspect of the invention, the present invention provides an expression vector comprising a nucleic acid molecule of present invention operatively linked to regulatory elements. Preferably, the regulatory elements include an inducible promoter.

In another aspect of the invention, the present invention provides a host cell transformed with an expression vector of the present invention.

In a further aspect of the invention, the present invention provides an isolated Smac peptide or polypeptide comprising, consisting essentially of or consisting of an amino acid sequence having at least two contiguous amino acid residues derived from at least residues 56-139 of SEQ ID NO:1 and of which up to 184 contiguous amino acid residues can be derived from residues 56-239 of SEQ ID NO:1, a functional variant of each or a functional equivalent of each, each of which is capable of specifically binding to at least a portion of an Inhibitor of Apoptosis protein. In one embodiment, the encoded peptide or polypeptide has an amino acid sequence of at least the amino acids Ala-Val. In another embodiment, the encoded peptide or polypeptide has an amino acid sequence provided in SEQ ID NO:13.

In a still further aspect of the invention, the present invention provides a method for inducing apoptosis in a cell, comprising contacting the cell with at least one component selected from the group consisting of a peptide or polypeptide of the present invention and a nucleic acid molecule of the present invention, under conditions and for a time sufficient to permit the induction of apoptosis in the cell. The cell can be a neoplastic or tumor cell, especially where the cell overexpresses an inhibitor of a caspase. Preferably, the inhibitor inhibits the activation of activity of caspase-3, caspase-7 and/or caspase-9 and the inhibitor is an IAP.

Another aspect of the present invention provides for an antibody that specifically binds to a peptide or polypeptide of the present invention. In a related aspect, the invention provides for an antibody that specifically binds to an epitope located on the N-terminus of Smac. In certain embodiments, the antibody inhibits the binding of Smac to at least a portion of an IPA. Preferably the portion of the IPA is at least one BIR domain, e.g., BIR1, BIR2 and/or BIR3, or it can be a full-length IAP. In a further embodiment, the antibody binds to an epitope that includes the amino acid sequence provided in SEQ ID NO:13.

An additional aspect of the present invention provides for a composition comprising a nucleic acid molecule of the present invention, a peptide of the present invention or an antibody of the present invention; and a physiologically acceptable carrier.

It is another aspect of the invention, the present invention provides a method for identifying an inhibitor or enhancer of a caspase-mediated apoptosis. This method comprises (a) contacting a cell transformed or transfected with a vector expressing a Smac peptide or polypeptide according to the present invention with a candidate inhibitor or candidate enhancer; and (b) detecting cell viability. An increase in cell viability indicates the presence of an inhibitor and a decrease in cell viability indicates the presence of an enhancer.

In yet another aspect of the present invention, the invention provides another method for identifying an inhibitor or enhancer of the caspase-mediated apoptosis process. This method comprises (a) contacting a cell transformed or transfected with a vector expressing Smac peptide or polypeptide of the present invention with a candidate inhibitor or candidate enhancer; and (b) detecting the presence of large and small caspase subunits, and therefrom determining the level of caspase processing activity. A decrease in the processing of the procaspase indicates the presence of an inhibitor and an increase in processing indicates the presence of an enhancer. Preferably the caspase detected is caspase-3, caspase-7 and/or caspase-9.

In yet a further aspect of the present invention, the invention provides method for identifying a compound that inhibits apoptosis. This method comprises: (a) separately contacting a plurality of cell populations expressing a cytosolic Smac and an inhibitor of BID with a compound to be tested for apoptotic inhibiting activity; (b) incubating said cell populations with a direct stimulus of the cell death pathway; and (c) measuring the specific apoptotic activity of the cell populations. The inhibition of the specific apoptotic activity is indicative that the compound is an inhibitor of apoptosis. Preferably, the direct stimulus of the cell death pathway is selected from the group consisting of Fas ligand, anti-Fas antibody and staurosporine UV and gamma irradiation. Part (c) can further comprise lysing said cells and determining caspase activity in said lysate. The compound may exhibits caspase inhibitory activity, inhibits apoptosis by promoting the activity of a cell survival polypeptide and/or exhibits cell death polypeptide inhibitory activity.

In another aspect of the present invention, the invention provides in vitro assays for identifying a compound that inhibits Smac binding to a Smac-binding molecule. This method comprises contacting a candidate compound with a Smac peptide in the presence of a Smac-binding molecule; and detecting displacement or inhibition of binding of said Smac-binding molecule from said Smac peptide or performing a functional assay that confirms displacement of said Smac-binding molecule from said Smac peptide. In one embodiment, the Smac-binding molecule is at least a portion of an IAP. Preferably the portion of the IPA is at least one BIR domain, e.g., BIR1, BIR2 and/or BIR3, or it can be a full-length IAP. In one embodiment, the functional assay detects the presence of large and small caspase subunits, and therefrom determining the level of caspase processing activity, wherein a decrease in processing confirms displacement. Preferably, the caspase detected is caspase-3, caspase-7 and/or caspase-9. In another embodiment, the functional assay detects the presence of a substrate cleavage product produced by a caspase cleavage of a substrate. An example of a substrate that can be used in this functional assay is acetyl DEVD-aminomethyl coumarin (DEVD-AMC).

In a further aspect of the present invention, the inventions provides for an isolated nucleic acid molecule comprising, consisting essentially of or consisting of a polynucleotide having a sequence encoding a cytosolic isoform of Smac as well as for an isolated polypeptide having an amino acid sequence of a cytosolic isoform of Smac.

These and other aspects of the present invention will become evident upon reference to the following detailed description and attached drawings. In addition, the various references set forth herein describe more detail certain procedures or composition (e.g., plasmids, etc.) and therefore incorporated by reference.

### BRIEF DESCRIPTION OF THE DRAWINGS

FIG. 1 is a schematic representation of the N-termini of the Smac precursor (Smac-L) and the alternatively spliced short isoform of Smac (Smac-S).

FIG. 2 depicts reverse transcriptase-polymerase chain reaction analysis of the expression of Smac-L (lanes 1) and Smac-S (lanes 2) in the following cell lines: Jurkat, 293, THP1, MCF7, A431 and 697.

FIGS. 3A-3C show photographs of MCF-7 cells transfected with constructs encoding GFP (3A) or C-terminal GFP-tagged Smac-S (3B) or C-terminal GFP-tagged Smac-L (3C) and visualized by confocal microscopy twenty-four hours after transfection.

FIG. 4 is a scanned image of an autoradiogram representing SDS-PAGE analysis of ³⁵S-labeled procaspase-9 (upper panel) or procaspase-3 (lower panel) processing in the presence of XIAP, cytochrome, dATP and increasing of purified mature Smac (50, 100 or 200 nM) or Smac-S (50, 100, 200 or 500 nM).

FIG. 5 is a bar graph representation of the percentage of the activity of caspase-3 or -7 in the absence of XIAP (100%) following the mixing of purified caspase-3 or caspase-7 with XIAP (20 nM in case of caspase-3, 5 nM in case of caspase-7), incubating the mixture with increasing amounts of purified mature Smac or Smac-S (100, 200, 500 or 1000 nM, respectively) in the presence of the peptide substrate DEVD-AMC (50 μM) for thirty minutes, and measuring the substrate cleavage by luminescence spectrometry.

FIGS. 6A and 6B present the interaction of XIAP and its isolated BIR domains with His6-tagged mature Smac or His6-tagged Smac-S. FIG. 6A illustrates XIAP and its isolated BIR domains used in this experiment. FIG. 6B is a scanned image of an autoradiogram representing SDS-PAGE analysis of the interaction of His6-tagged mature Smac (lanes 2, 5, 8) or His6-tagged Smac-S (lanes 3, 6, 9) with ³⁵S-labeled XIAP, or BIR1/BIR2 or BIR3/RING domains of XIAP.

FIGS. 7 is a schematic diagram of mature Smac and N-terminal and C-terminal deletion mutants of Smac used in the experiments, the results of which are depicted in FIGS. 8-10.

FIGS. 8A and 8B are a scanned images of an autoradiogram representing SDS-PAGE analysis of ³⁵S-labeled procaspase-3 processing in the presence of XIAP, cytochrome c, dATP and increasing amounts of the N-terminal mutants (100, 200 or 500 nM) (8A) or the C-terminal mutants (100, 200, 500 or 1000 nM) (8B).

FIGS. 9A-9D are bar graph representations of the percentage of the activity of caspase-3 or -7 in the absence of XIAP (100%) following the mixing of purified caspase-3 (9A and 9C) or caspase-7 (9B and 9D) with XIAP, incubating the mixture with increasing amounts of purified N-terminal deletion mutants (9A and 9B) or C-terminal deletion mutants (9C and 9D) (100, 200, 500 or 1000 nM, respectively in the presence of the peptide substrate DEVD-AMC, and measuring the substrate cleavage by luminescence spectrometry.

FIG. 10 is a scanned image of an autoradiogram representing SDS-PAGE analysis of the interaction of His6-tagged N-terminal and C-terminal deletion mutants with ³⁵S-labeled XIAP, or BIR1/BIR2 or BIR3/RING domains of XIAP.

FIG. 11 is a bar graph representation of the effect of Smac N-terminal peptides on cytochrome c-mediated caspase-3 activation. Purified caspase-3 was mixed with purified XIAP (20 nM) and then stimulated with cytochrome c plus dATP in the presence of increasing amounts of Smac (25, 100, 500 nM) or the indicated purified N-terminal Smac or Smac-S peptides (25, 100, 500 μM). The reactions were carried out in the presence of DEVD-AMC as a substrate and measured the substrate cleavage by luminescence spectrometry. Peptide-1 was AVPIAQK (SEQ ID NO:6); Peptide-2 was MKSDFYFQK (SEQ ID NO:9); Peptide-3 was TDSTSTFL (SEQ ID NO:10); Peptide-4 was AVPIAQKSEPHSLSSEALMRRAVSLVTDSTSTFLS (SEQ ID NO:11).

FIG. 12 schematic diagram of a GFP-Smac fusion protein and its cleavage by caspase-8 to generate a mature cytosolic Smac.

FIG. 13 is a bar graph representation of the effect of expression of cytosolic Smac on TRAIL-induced apoptosis of MCF-7 cells transfected with GFP of GFP-Smac expression constructs together with equal amounts of empty vector or a construct encoding Bcl-xL.

FIG. 14 presents a model of cross talk between the death receptor pathway and the mitochondrial pathway, and the role of Smac in neutralizing the inhibitory effect of XIAPs on the initiator and effector caspases.

### DETAILED DESCRIPTION OF THE INVENTION

Prior to setting forth the invention, it may be helpful to an understanding thereof to set forth definitions of certain terms that will be used hereinafter.

An “isolated nucleic acid molecule” refers to a polynucleotide molecule in the form of a separate fragment or as a component of a larger nucleic acid construct, which has been separated from its source cell (including the chromosome it normally resides in) at least once, and preferably in a substantially pure form. Nucleic acid molecules may be comprised of a wide variety of nucleotides, including DNA, RNA, nucleotide analogues, or a combination thereof.

A “functional” peptide or polypeptide, as used herein, refers to a peptide or polypeptide derived from at least the N-terminal domain of the Smac polypeptide that retains at least one biological or functional activity associated with N-terminal domain of Smac. Preferably the biological or functional activity is the specific binding to at least a portion of an Inhibitor of Apoptosis Protein (IAP). Preferably this portion of the IPA to which the peptide or polypeptide specifically binds is a BIR domain. In one embodiment this BIR domain is BIR1. In another embodiment this BIR domain is BIR2. In a further embodiment this BIR domain is BIR3. In certain embodiments, the peptide or polypeptide specifically binds to more than one BIR domain or to a full length IAP. Such functional peptides or polypeptides may comprise, consist essentially of or consist of an amino acid sequence having at least two, preferably four, contiguous amino acids derived from at least residues 56-139 of SEQ ID NO:1. In those embodiments where the peptide has only two contiguous amino acids, the preferred amino acids are Ala-Val (AV). An example of an embodiment with four contiguous amino acids is AVPI (SEQ ID NO:13).

As used herein, a “peptide” is an amino acid sequence of between two and ten contiguous amino acids, including all integer values in between, e.g., 2, 4, 5, 6, 7, 8, 9 and 10 contiguous amino acids. A “polypeptide” is an amino acid sequence of more than ten contiguous amino acids up to and including “mature” Smac, including all integer values in between, e.g., 11, 15, 20, 30, 40, 60, 75, 100, 125, 150, 160, 175, 190, 200 or more contiguous amino acids. “Mature” Smac is a Smac polypeptide without the 55 amino acid residue mitochondrial targeting sequence (MTS), residues 1-55 of SEQ ID NO:1. “Cytosolic” Smac or “short” Smac (Smac-S) is a Smac isoform that begins with the MKSDFYF sequence (SEQ ID NO:4), which replaces the MTS and residues 56-60 (AVPIA-SEQ ID NO:5) of SEQ ID NO:1, the long Smac isoform (Smac-L), i.e., mature Smac with the MTS included). Smac-S and Smac-L are 100% identical after residue 60 of SEQ ID NO:1.

A functional equivalent of a Smac peptide or polypeptide is a peptide or polypeptide with at least one amino acid substitution and retains at least one functional activity associated with N-terminal domain of Smac. Preferably the functional activity is the specific binding to at least a portion of an IAP. For example, an Ala-Thr (AT) peptide is a functional equivalent for the AV peptide and can substitution for AV in any peptide or polypeptide with AV at its N-terminus; and an ATPF (SEQ ID NO:14), an AVAF (SEQ ID NO:15), an AVPF (SEQ ID NO:16), an AVPY (SEQ ID NO:17) and an ATPV (SEQ ID NO:18) is a functional equivalent for the AVPI peptide (SEQ ID NO:13) and can substitution for AVPI (SEQ ID NO:13) in any peptide or polypeptide with AVPI (SEQ ID NO:13) at its N-terminus.

References to Smac herein are intended to include peptides of any origin which are substantially homologous to and which are biologically or function equivalent to the Smac peptides and polypeptides characterized and described herein.

A “caspase” refers to a cysteine protease that specifically cleaves proteins after Asp residues. Caspases are initially expressed as zymogens, in which a large subunit is N-terminal to a small subunit. Caspases are generally activated by cleavage at internal Asp residues. These proteins have been identified in many eukaryotes, including C. elegans, Drosophila, mouse, and humans. Currently, there are at least 14 known caspase genes, named caspase-1 through caspase-14. Caspases are found in myriad organisms, including human, mouse, insect (e.g., Drosophila), and other invertebrates (e.g., C. elegans). In Table 1, ten human caspases are listed along with their alternative names.

| Caspase | Alternative name |
|---|---|
| Caspase-1 | ICE |
| Caspase-2 | ICH-1 |
| Caspase-3 | CPP32, Yama, apopain |
| Caspase-4 | ICEᵣₑₗII; TX, ICH-2 |
| Caspase-5 | ICEᵣₑₗIII; TY |
| Caspase-6 | Mch2 |
| Caspase-7 | Mch3, ICE-LAP3, CMH-1 |
| Caspase-8 | FLICE; MACH; Mch5 |
| Caspase-9 | ICE-LAP6; Mch6 |
| Caspase-10 | Mch4, FLICE-2 |

The term “in vitro” refers to cell free systems.

The current invention includes compositions comprising nucleic acids encoding and peptides and polypeptides corresponding to a peptide of Smac or variants thereof that retains at least one functional activity associated with N-terminal domain of Smac. In one embodiment, a peptide or polypeptide has at least two contiguous amino acid residues derived from at least residues 56-139 of SEQ ID NO:1. In addition, the invention identifies methods of using the peptides of the invention for apoptosis modulation and to identify modulators of a caspase-mediated apoptosis as well as in therapeutic uses.

A. Smac Nucleic Acid Molecules

The present invention provides nucleic acid molecules that encode peptides of Smac or variants thereof. In one embodiment, the encoded peptide or variants have at least four contiguous amino acids derived from residues 56-139 of SEQ ID NO:1. The invention includes any and all nucleic acid sequences that encode this Smac peptide or variant thereof. Smac peptides may be identified as such by any means known in the art, including sequence and functional analysis. Preferably the Smac peptide or variant has the ability to bind an Inhibitor of Apoptosis protein (IAP) or a portion of an IAP. In certain embodiments the portion of the IAP is a BIR1, a BIR2 and/or a BIR3 domain.

The nucleic acid sequence for full-length Smac is available in GenBank/EBI DataBank at Accession No. AF262240. The short isoform of Smac of one embodiment of the current invention were identified as described in Example 1 and the sequence for this isoform has been submitted to the GenBank/EBI DataBank and is available at Accession No. AF298770.

Smac nucleic acid molecules may be isolated from genomic DNA or cDNA according to practices known in the art (see Sambrook et al., Molecular Cloning: A Laboratory Manual, Cold Spring Harbor Press, 1989).

Other methods may also be utilized to obtain Smac nucleic acid molecules. One preferred method is to perform polymerase chain reaction (PCR) to amplify a Smac nucleic acid molecule from cDNA or genomic DNA using oligonucleotide primers corresponding to the 5′ and 3′ ends of Smac nucleic acid molecules or regions thereof. Detailed methods of PCR cloning may be found in Ausubel, et al., Current Protocols in Molecular Biology, Greene Publishing Associates and Wiley-Interscience, New York, 1995, for example.

Polynucleotides of the invention may also be made using the techniques of synthetic chemistry given the sequences disclosed herein. The degeneracy of the genetic code permits alternate nucleotide sequences that will encode the same amino acid sequences. All such nucleotide sequences are within the scope of the present invention.

Nucleic acid sequences encoding Smac peptides may be fused to sequences encoding a secretion signal or sequences encoding the MTS sequence can be removed, whereby the resulting polypeptide is a precursor protein that is subsequently processed and secreted. The resulting processed Smac polypeptide may be recovered from the cell lysate, periplasmic space, phloem, or from the growth or fermentation medium. Secretion signals suitable for use are widely available are well known in the art (e.g., von Heijne, J. Mol. Biol. 184:99-105, 1985).

The Smac nucleic acid molecules of the subject invention also include variants (including alleles) of the native nucleic acid molecules of the present invention. Variants of the Smac nucleic acid molecules provided herein include natural variants (e.g., polymorphisms, splice variants or mutants) and those produced by genetic engineering (e.g, substitutions, deletions or addition of residues). Many methods for generating mutants have been developed (see generally, Ausubel et al., supra). Preferred methods include alanine scanning mutagenesis and PCR generation of mutants using an oligonucleotide containing the desired mutation to amplify mutant nucleic acid molecules. Variants generally have at least 70% or 75% nucleotide identity to the native sequence, preferably at least 80%-85%, and most preferably at least 90%, 92%, 93%, 94%, 95%, 96%, 97%, 98% or 99% nucleotide identity. The identity algorithms and settings that may be used are defined herein infra, but may also include using computer programs which employ the Smith-Waterman algorithm, such as the MPSRCH program (Oxford Molecular), using an affine gap search with the following parameters: a gap open penalty of 12 and a gap extension penalty of 1. A preferred method of sequence alignment uses the GCG PileUp program (Genetics Computer Group, Madison, Wis.) (Gapweight: 4, Gaplength weight: 1). In certain embodiments the alignment algorithm utilizes default parameters. Further, a nucleotide variant will typically be sufficiently similar in sequence to hybridize to the reference sequence under moderate or stringent hybridization conditions. For nucleic acid molecules over about 500 bp, stringent conditions include a solution comprising about 1 M Na⁺ at 25° to 30° C. below the Tm; e.g., 5×SSPE, 0.5% SDS, at 65° C.; see Ausubel et al., Current Protocols in Molecular Biology, Greene Publishing, 1995; Sambrook et al., Molecular Cloning: A Laboratory Manual, Cold Spring Harbor Press, 1989). Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

Typically, for stringent hybridization conditions a combination of temperature and salt concentration should be chosen that is approximately 12-20° C. below the calculated Tm of the hybrid under study. The Tm of a hybrid between a nucleotide sequence of the present invention and a polynucleotide sequence which is 70%, 75%, 80%, 85%, 90%, 95%, 96%, 97%, 98%, or 99% identical can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(% G+C)−0.63(% formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C. Suitable moderately stringent conditions include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

Nucleic acid sequences which are substantially the same as the nucleic acid sequences encoding Smac are included within the scope of the invention. Such substantially same sequences may, for example, be substituted with codons optimized for expression in a given host cell such as E. coli. The present invention also includes nucleic acid sequences that will hybridize to sequences that encode viral, human, or murine Smac or complements thereof. The invention includes nucleic acid sequences encoding peptides and polypeptides of at least the N-terminus of the Smac protein. Deletions, insertions and/or nucleotide substitutions within a Smac nucleic acid molecule are also within the scope of the current invention. Such alterations may be introduced by standard methods known in the art such as those described in Ausubel et al., supra. Also included are nucleic acid sequences encoding functional equivalents of a Smac peptide or polypeptide. In addition, the invention includes nucleic acids that encode polypeptides that are recognized by antibodies that bind a Smac peptide, polypeptide, functional variants of each and functional equivalents of each.

Polynucleotide molecules of the invention can comprise at least 9, 12, 15, 18, 21, 24, 27, 30, 33, 36, 39, 42, 54, 60, 66, 72, 84, 90, 100, 120, 140, 200, 240, 250, 300, 330, 400, 420, 500 or more contiguous nucleotides derived from nucleotide position 185 up to and including nucleotide position 736 of SEQ ID NO:1 or the complements thereof.

Polynucleotide molecules of the invention also include molecules which encode single-chain antibodies which specifically bind to the disclosed peptides that specifically bind to mRNA encoding the disclosed proteins, and fusion proteins comprising amino acid sequences of the disclosed proteins.

B. Smac Peptides

The present invention includes that in one embodiment the polypeptide or peptide sequences are derived from at least the N-terminus of Smac but not including the full length sequence of Smac or Smac-L. In certain embodiments, the peptides comprise, consist essentially of or consist of at least two contiguous amino acid residues derived from at least residues 56-139 of SEQ ID NO:1 and that specifically bind to a portion of an Inhibitor of Apoptosis Protein (IAP) (e.g., XIAP and CIAP) or to a full length IAP. In other embodiments, such functional peptides or polypeptide comprise, consist essentially of or consist of at least 2 contiguous amino acids derived from residues 56-139 of SEQ ID NO:1 and that up to 184 contiguous amino acids can be derived from residues 56-239 of SEQ ID NO:1 (i.e., mature Smac) including all integer values in between, e.g., 2, 4, 5, 7, 9, 10, 15, 20, 30, 40, 50, 60, 70, 80, 90, 100, 110, 120, 125, 130, 140, 150, 155, 160, 170, 180 or more contiguous amino acids and have at least about 75% or 80% amino acid sequence identity with a peptide derived from residues 56-185 of SEQ ID NO:1. In other embodiments, such a functional peptide or polypeptide comprises at least 2 to 185 contiguous amino acids of SEQ ID NO:1 including all integer values in between or more contiguous amino acids and have at least about 85%, 90%, 92%, 95%, 97%, 98% or 99% amino acid sequence identity with a peptide or polypeptide derived from at least residues 56-139 of SEQ ID NO:1. The aforementioned identities may be calculated with any one of the algorithms herein described.

The current invention encompasses all variants (including alleles) of the native Smac peptide or polypeptide sequences as defined in the present invention that retains at least one biological or functional activity associated with N-terminal domain of Smac. Preferably the biological or functional activity is the specific binding to at least a portion of an Inhibitor of Apoptosis Protein (IAP). Such functional variants may result from natural polymorphisms or may be synthesized by recombinant methodology, and differ from wild-type peptides by one or more amino acid substitutions, insertions, deletions, or the like. Amino acid changes in functional variants of Smac peptides or polypeptides may be conservative substitutions. Guidance in determining which amino acid residues can be substituted, inserted, or deleted without abolishing biological or immunological activity can be found using computer programs well known in the art, such as DNASTAR software. Preferably, amino acid changes in secreted functional variants are conservative amino acid changes, i.e., substitutions of similarly charged or uncharged amino acids. It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant. Whether an amino acid change results in a functional secreted protein or polypeptide can readily be determined by testing the altered protein or polypeptide in a functional assay, for example, as disclosed in U.S. Pat. No. 5,654,173 and described in detail below.

A conservative amino acid change involves substitution of one amino acid for another amino acid of a family of amino acids with structurally related side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified as aromatic amino acids. Non-naturally occurring amino acids can also be used to form protein variants of the invention.

In the region of homology to the native sequence, functional variants should preferably have at least 70-99% amino acid identity, including all integer values in between, e.g., at least 70%, 75%, 80%, 90%, 92%, 95%, 97%, 98% or 99% amino acid identity. In certain embodiments the peptide or polypeptide sequence is compared to a test sequence or when necessary a particular domain is compared to a test sequence to determine percent identity. Typically by utilizing default parameters. Such amino acid sequence identity may be determined by standard methodologies, including those set forth supra as well as the use of the National Center for Biotechnology Information BLAST 2.0 search methodology (Altschul et al., J. Mol. Biol. 215:403-10, 1990). In one embodiment BLAST 2.0 is utilized with default parameters. A preferred method of sequence alignment uses the GCG PileUp program (Genetics Computer Group, Madison, Wis.) (Gapweight: 4, Gaplength weight: 1). The pileUp program creates a multiple sequence alignment form a group of related sequences using progressive, pairwise alignments. PileUp creates a multiple sequence alignment using the progressive alignment method of Feng and Doolittle (J. Mol. Evol. 25:351-360, 1987) and is similar to the method described by Higgins and Sharp (CABIOS 5:151-153, 1989). Further, whether an amino acid change results in a functional peptide can be readily determined by assaying biological properties of the disclosed peptides. For example, the biological properties of Smac functional variants can be assayed by determining whether they bind to at least a portion of a IAP, as described in Example 2, or by examining their effects of apoptosis and/or caspase activation, as described in Examples 4 and 5.

Smac functional variants can include hybrid and modified forms of Smac peptides or polypeptides such as, but not limited to, fusion polypeptides. Smac fusion polypeptides include peptides or polypeptides of Smac fused to amino acid sequences comprising one or more heterologous polypeptides. Such heterologous polypeptides may correspond to naturally occurring polypeptides of any source or may be recombinantly engineered amino acid sequences. Fusion proteins are useful for purification, generating antibodies against amino acid sequences, and for use in various assay systems. For example, fusion proteins can be used to identify proteins or a domain of that protein which interacts with a peptide or polypeptide of the invention or which interferes with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of one or more of the disclosed proteins can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can be selected from any contiguous amino acid sequences as herein described.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex A DNA binding domain (DBD) fusions, GAL4 DNA binding domain fusions, and herpes simplex virus (HSV) BP16 protein fusions.

These fusions can be made, for example, by covalently linking two protein segments or by standard procedures in the art of molecular biology. Recombinant DNA methods can be used to prepare fusion proteins, for example, by making a DNA construct which comprises coding sequences derived from SEQ ID NO:1 in proper reading frame with nucleotides encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

These heterologous polypeptides may be of any length and may include one or more amino acids. In certain embodiments, Smac fusion proteins may be produced to facilitate expression or purification. For example, a Smac polypeptide may be fused to maltose binding protein or glutathione-S-transferase. In other embodiments, Smac fusion proteins may contain an epitope tag to facilitate identification or purification. One example of a tag is the FLAG epitope tag (Kodak). Smac variants may have certain amino acids which have been deleted, replaced or modified. Variants can also include post-translational modifications.

C. Vectors, Host Cells and Means of Expressing and Producing Protein

The present invention encompasses vectors comprising regulatory elements linked to Smac nucleic acid sequences. Such vectors may be used, for example, in the propagation and maintenance of Smac nucleic acid molecules or the expression and production of Smac peptides or polypeptides or functional variants of each or functional equivalents of each and nucleic acid molecules. Vectors may include, but are not limited to, plasmids, episomes, baculovirus, retrovirus, lentivirus, adenovirus, and parvovirus including adeno-associated virus.

Smac may be expressed in a variety of host organisms. In certain embodiments, Smac is produced in mammalian cells, such as CHO, COS-7, or 293 cells. Other suitable host organisms include bacterial species (e.g., E. coli and Bacillus), other eukaryotes such as yeast (e.g., Saccharomyces cerevisiae), plant cells and insect cells (e.g., Sf9). Vectors for these hosts are well known in the art.

A DNA sequence encoding Smac or is introduced into an expression vector appropriate for the host. The sequence is derived from an existing clone or synthesized. As described herein, a fragment of the coding region may be used. A preferred means of synthesis is amplification of the nucleic acid molecule encoding the peptide of the present invention from cDNA, genomic DNA, or a recombinant clone using a set of primers that flank the desired portion of the protein. Restriction sites are typically incorporated into the primer sequences and are chosen with regard to the cloning site of the vector. If necessary, translational initiation and termination codons can be engineered into the primer sequences. The sequence of Smac can be codon-optimized for expression in a particular host. For example, a Smac isolated from a human cell that is expressed in a fungal host, such as yeast, can be altered in nucleotide sequence to use codons preferred in yeast. Further, it may be beneficial to insert a traditional AUG initiation codon at the CUG initiation positions to maximize expression, or to place an optimized translation initiation site upstream of the CUG initiation codon. Accordingly, such codon-optimization may be accomplished by methods such as splice overlap extension, site-directed mutagenesis, automated synthesis, and the like.

At minimum, the vector must contain a promoter sequence. As used herein, a “promoter” refers to a nucleotide sequence that contains elements that direct the transcription of a linked gene. At minimum, a promoter contains an RNA polymerase binding site. More typically, in eukaryotes, promoter sequences contain binding sites for other transcriptional factors that control the rate and timing of gene expression. Such sites include TATA box, CAAT box, POU box, AP1 binding site, and the like. Promoter regions may also contain enhancer elements. When a promoter is linked to a gene so as to enable transcription of the gene, it is “operatively linked”.

Typical regulatory elements within vectors include a promoter sequence that contains elements that direct transcription of a linked gene and a transcription termination sequence. The promoter may be in the form of a promoter that is naturally associated with the gene of interest. Alternatively, the nucleic acid may be under control of a heterologous promoter not normally associated with the gene. For example, tissue specific promoter/enhancer elements may be used to direct expression of the transferred nucleic acid in repair cells. In certain instances, the promoter elements may drive constitutive or inducible expression of the nucleic acid of interest. Mammalian promoters may be used, as well as viral promoters capable of driving expression in mammalian cells. Examples of other regulatory elements that may be present include secretion signal sequences, origins of replication, selectable markers, recombinase sequences, enhancer elements, nuclear localization sequences (NLS) and matrix association regions (MARS).

The expression vectors used herein include a promoter designed for expression of the proteins in a host cell (e.g., bacterial). Suitable promoters are widely available and are well known in the art. Inducible or constitutive promoters are preferred. Such promoters for expression in bacteria include promoters from the T7 phage and other phages, such as T3, T5, and SP6, and the trp, lpp, and lac operons. Hybrid promoters (see U.S. Pat. No. 4,551,433), such as tac and trc, may also be used. Promoters for expression in eukaryotic cells include the P10 or polyhedron gene promoter of baculovirus/insect cell expression systems (see, e.g., U.S. Pat. Nos. 5,243,041, 5,242,687, 5,266,317, 4,745,051, and 5,169,784), MMTV LTR, CMV IE promoter, RSV LTR, SV40, metallothionein promoter (see, e.g., U.S. Pat. No. 4,870,009) and the like.

The promoter controlling transcription of Smac may itself be controlled by a repressor. In some systems, the promoter can be derepressed by altering the physiological conditions of the cell, for example, by the addition of a molecule that competitively binds the repressor, or by altering the temperature of the growth media. Preferred repressor proteins include, but are not limited to, the E. coli lacI repressor responsive to IPTG induction, the temperature sensitive λcI857 repressor, and the like. The E. coli lacI repressor is preferred.

In other preferred embodiments, the vector also includes a transcription terminator sequence. A “transcription terminator region” has either a sequence that provides a signal that terminates transcription by the polymerase that recognizes the selected promoter and/or a signal sequence for polyadenylation.

Preferably, the vector is capable of replication in the host cells. Thus, when the host cell is a bacterium, the vector preferably contains a bacterial origin of replication. Preferred bacterial origins of replication include the f1-ori and col E1 origins of replication, especially the ori derived from pUC plasmids. In yeast, ARS or CEN sequences can be used to assure replication. A well-used system in mammalian cells is SV40 ori.

The plasmids also preferably include at least one selectable marker that is functional in the host. A selectable marker gene includes any gene that confers a phenotype on the host that allows transformed cells to be identified and selectively grown. Suitable selectable marker genes for bacterial hosts include the ampicillin resistance gene (Amp^{r}), tetracycline resistance gene (Tc^{r}) and the kanamycin resistance gene (Kan^{r}). The kanamycin resistance gene is presently preferred. Suitable markers for eukaryotes usually require a complementary deficiency in the host (e.g., thymidine kinase (tk) in tk-hosts). However, drug markers are also available (e.g., G418 resistance and hygromycin resistance).

The sequence of nucleotides encoding Smac may also include a secretion signal or the mitochondrial targeting sequence (MTS) sequence can be removed, whereby the resulting peptide or polypeptide is a precursor protein processed and secreted. The resulting processed peptide or polypeptide may be recovered from the periplasmic space, the growth medium, phloem, etc. Secretion signals suitable for use are widely available and are well known in the art (von Heijne, J. Mol. Biol. 184:99-105, 1985). Prokaryotic and eukaryotic secretion signals that are functional in E. coli (or other host) may be employed. The presently preferred secretion signals include, but are not limited to, those encoded by the following E. coli genes: pelB (Lei et al., J. Bacteriol. 169:4379, 1987), phoA, ompA, ompT, ompF, ompC, beta-lactamase, and alkaline phosphatase.

One skilled in the art appreciates that there are a wide variety of suitable vectors for expression in bacterial cells that are readily obtainable. Vectors such as the pET series (Novagen, Madison, Wis.), the tac and trc series (Pharmacia, Uppsala, Sweden), pTTQ18 (Amersham International plc, England), pACYC 177, the pGEX series, and the like are suitable for expression of Smac. Baculovirus vectors, such as pBlueBac (see, e.g., U.S. Pat. Nos. 5,278,050, 5,244,805, 5,243,041, 5,242,687, 5,266,317, 4,745,051, and 5,169,784; available from Invitrogen, San Diego) may be used for expression in insect cells, such as Spodoptera frugiperda sf9 cells (see U.S. Pat. No. 4,745,051). The choice of a bacterial host for the expression of Smac is dictated in part by the vector. Commercially available vectors are paired with suitable hosts.

A wide variety of suitable vectors for expression in eukaryotic cells are also available. Such vectors include pCMVLacI, pXT1 (Stratagene Cloning Systems, La Jolla, Calif.); pCDNA series, pREP series, pEBVHis (Invitrogen, Carlsbad, Calif.). In certain embodiments, Smac gene is cloned into a gene targeting vector, such as pMC1neo, a pOG series vector (Stratagene Cloning Systems).

The Smac peptides or polypeptides may be isolated by standard methods, such as affinity chromatography, size exclusion chromatography, metal ion chromatography, ionic exchange chromatography, HPLC, and other known protein isolation methods. (See generally Ausubel et al., supra; Sambrook et al., supra). An isolated purified peptide or polypeptide usually gives a single band on SDS-PAGE when stained with Coomassie blue.

The Smac peptides or polypeptides, as discussed earlier, may be expressed as fusion proteins to aid in purification. Such fusions may be, for example, glutathione-S-transferase fusions, Hex-His fusions, or the like such that the fusion construct may be easily isolated. With regard to Hexa-His fusions, such fusions can be isolated by metal-containing chromatography, such as nickel-coupled beads. Briefly, a sequence encoding His₆ is linked to a DNA sequence encoding Smac. Although the His₆ sequence can be positioned anywhere in the molecule, preferably it is linked at the 3′ end immediately preceding the termination codon. The fusion may be constructed by any of a variety of methods. A convenient method is amplification of the Smac gene using a downstream primer that contains the codons for His₆.

The purified Smac peptide or polypeptide may be used in various assays to screen for modulators (i.e., inhibitors or enhancers) of apoptosis. These assays may be performed in vitro or in vivo and utilize any of the methods described herein or that are known in the art. The protein may also be crystallized and subjected to X-ray analysis to determine its 3-dimensional structure. The Smac peptides may also be used as immunogens for raising antibodies.

Recombinant Smac peptides or polypeptides may be produced by expressing the DNA sequences provided in the invention. Using methods known in the art, a Smac peptide or polypeptide expression vector may be constructed, transformed into a suitable host cell, and conditions suitable for expression of a Smac peptide by the host cell established. One skilled in the art will appreciate that there are a wide variety of suitable vectors for expression in bacterial cells (e.g. pET series (Novagen, Madison, Wis.)), insect cells (e.g. pBlueBac (Invitrogen, Carlsbad, Calif.)), and eukaryotic cells (e.g. pCDNA and pEBVHis (Invitrogen, Carlsbad, Calif.)). In certain embodiments, the Smac nucleic acid molecule may be cloned into a gene targeting vector such as pMC1neo (Stratagene, La Jolla, Calif.). Synthetic chemistry methods, such as solid phase peptide synthesis can also be used to synthesize proteins, fusion proteins, or polypeptides of the invention.

The resulting expressed peptide or polypeptide can be purified from the culture medium or from extracts of the cultured cells. Methods of protein purification such as affinity chromatography, ionic exchange chromatography, HPLC, size exclusion chromatography, ammonium sulfate crystallization, electrofocusing, or preparative gel electrophoresis are well known and widely used in the art (see generally Ausubel et al., supra; Sambrook et al., supra). An isolated purified protein is generally evidenced as a single band on an SDS-PAGE gel stained with Coomassie blue.

D. Smac Antibodies

Antibodies to the Smac peptides or polypeptides or functional variants of each or functional equivalents of each are provided by the invention. Antibodies of the invention can be used, for example, to detect Smac peptides, polypeptides, variants of each or functional equivalents of each. The antibodies can be used for isolation of Smac peptides, polypeptides, variants of each or functional equivalents of each and in the identification of molecules that interact with Smac peptides, polypeptides, variants of each or functional equivalents of each. The antibodies may also be used to inhibit or enhance the biological activity of Smac peptides, polypeptides, functional variants of each or functional equivalents of each.

One such biological activity is the binding of the Smac peptides, polypeptides, functional variants of each or functional equivalents of each to at least a portion of an IAP or to a full length IAP. Preferably this portion of an IAP is at least one of the BIR domains, e.g. BIR1, BIR2 or BIR3. Accordingly, the antibodies can be specific for the N-terminus of Smac and/or inhibit the binding of the at least a portion of an IAP or the entire full length of an IAP to Smac. In one embodiment, an inhibiting antibody would be specific to an epitope that includes the amino acids AVPI (SEQ ID NO:13).

Within the context of the current invention, an antibody includes both polyclonal and monoclonal antibodies (mAb); primatized (e.g., humanized); murine; mouse-human; mouse-primate; and chimeric; and may be an intact molecule, a fragment thereof (such as scFv, Fv, Fd, Fab, Fab′ and F(ab)′₂ fragments), or multimers or aggregates of intact molecules and/or fragments; and may occur in nature or be produced, e.g., by immunization, synthesis or genetic engineering; an “antibody fragment,” as used herein, refers to fragments, derived from or related to an antibody, which bind antigen and which in some embodiments may be derivatized to exhibit structural features that facilitate clearance and uptake, e.g., by the incorporation of galactose residues. This includes, e.g., F(ab), F(ab)′₂, scFv, light chain variable region (V_{L}), heavy chain variable region (V_{H}), and combinations thereof.

Antibodies are generally accepted as specific for the Smac peptides if they bind with a K_{d} of greater than or equal to 10⁻⁷M, and preferably 10⁻⁸M. The affinity of an antibody can be readily determined by one of ordinary skill in the art (see Skatchard, Ann. N.Y. Acad. Sci. 51:660-672, 1949).

Antibodies may be produced by any of a variety of methods available to one of ordinary skill in the art. Detailed methods for generating antibodies are provided in Antibodies: A Laboratory Manual, Harlow and Lane (eds.), Cold Spring Harbor Laboratories, 1988, which is incorporated by reference.

A polyclonal antibody may be readily generated in a variety of animals such as rabbits, mice and rats. Generally, an animal is immunized with a Smac peptide or one or more peptides comprising SMAC amino acid sequences which may be conjugated to a carrier protein. Routes of administration include intraperitoneal, intramuscular, intraocular, or subcutaneous injections, usually in an adjuvant (e.g., Freund's complete or incomplete adjuvant).

Monoclonal antibodies may be readily generated from hybridoma cell lines using conventional techniques (see Antibodies: A Laboratory Manual, Harlow and Lane (eds.), Cold Spring Harbor Laboratories, 1988). Various immortalization techniques such as those mediated by Epstein-Barr virus or fusion to produce a hybridoma may be used. In a preferred embodiment, immortalization occurs by fusion with a myeloma cell line (e.g., NS-1 (ATCC No. TIB 18) and P3X63-Ag 8.653 (ATCC No. CRL 1580)) to create a hybridoma that secretes a monoclonal antibody.

Antibody fragments, such as Fab and Fv fragments, may be constructed, for example, by conventional enzymatic digestion or recombinant DNA techniques to yield isolated variable regions of the antibody. Within one embodiment, the genes which encode the variable region from a hybridoma producing a monoclonal antibody of interest are amplified using nucleotide primers corresponding to the variable region. Amplification products are subcloned into plasmid vectors and propagated and purified using bacteria, yeast, plant or mammalian-based expression systems. Techniques may be used to change a murine antibody to a human antibody, known familiarly as a “humanized” antibody, without altering the binding specificity of the antibody.

Antibodies may be assayed for immunoreactivity against the Smac peptides by any of a number of methods, including western blot, enzyme-linked immuno-sorbent assays (ELISA), countercurrent immuno-electrophoresis, radioimmunoassays, dot blot assays, sandwich assays, inhibition or competition assays, or immunoprecipitation (see U.S. Pat. Nos. 4,376,110 and 4,486,530; see also Antibodies: A Laboratory Manual, Harlow and Lane (eds.), Cold Spring Harbor Laboratory Press, 1988). Techniques for purifying antibodies are those available in the art. In certain embodiments, antibodies are purified by passing the antibodies over an affinity column to which amino acid sequences of the present invention are bound. Bound antibody is then eluted. Other purification techniques include, but are not limited to HPLC or RP-HPLC, or purification on protein A or protein G columns.

A number of therapeutically useful molecules are known in the art that comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment which comprises both antigen-binding sites. An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A “humanized” antibody refers to an antibody derived from a non-human antibody (typically murine), or derived from a chimeric antibody, that retains or substantially retains the antigen-binding properties of the parent antibody but which is less immunogenic in humans. This may be achieved by various methods, including by way of example: (a) grafting only the non-human CDRs onto human framework and constant regions (humanization), or (b) transplanting the entire non-human variable domains, but “cloaking” them with a human-like surface by replacement of surface residues (“veneering”). Such methods are disclosed, for example, in Jones et al., Nature 321:522-525, 1986; Morrison et al., Proc. Natl. Acad. Sci. 81:6851-6855, 1984; Morrison and Oi, Adv. Immunol. 44:65-92, 1988; Verhoeyer et al., Science 239:1534-1536, 1988; Padlan, Molec. Immun. 28:489-498, 1991; Padlan, Molec. Immun. 31(3):169-217, 1994. In the present invention, humanized antibodies include “humanized” and “veneered” antibodies. A preferred method of humanization comprises alignment of the non-human heavy and light chain sequences to human heavy and light chain sequences, selection and replacement of the non-human framework with a human framework based on such alignment, molecular modeling to predict conformation of the humanized sequence and comparison to the conformation of the parent antibody, followed by repeated back mutation of residues in the CDR region which disturb the structure of the CDRs until the predicted conformation of the humanized sequence model closely approximates the conformation of the non-human CDRs of the parent non-human antibody.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

E. Methods of Using Smac Nucleic Acids and Peptides or Polypeptides

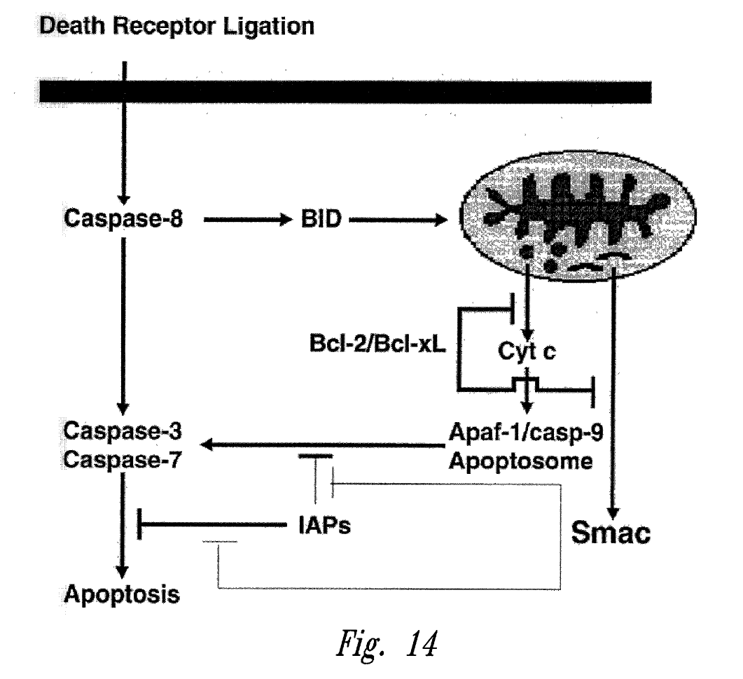

Smac peptides or polypeptides can induce apoptosis by interaction with the Inhibitors of Apoptosis proteins (IAPs)(see FIG. 14). Studies using the Smac peptides or a cytosolic Smac in the present application revealed that Smac is a key component of caspase-mediate apoptosis. Smac is capable of regulating or altering apoptosis. For example, a cytosolic Smac can be provided to type II cells and convert them to type I cells, so that death-receptor ligands can induce apoptosis. Thus, the compositions described herein, including Smac nucleic acids, peptides and antibodies, can be used for a variety of assays and for therapeutic purposes.

1. Identification of Inhibitors and Enhancers of Caspase-Mediated Apoptotic Activity

Candidate inhibitors and enhancers may be isolated or procured from a variety of sources, such as bacteria, fungi, plants, parasites, libraries of chemicals, peptides or peptide derivatives and the like. Inhibitors and enhancers may be also rationally designed, based on the protein structures determined from X-ray crystallography.

Without wishing to be bound to a particular theory or held to a particular mechanism, an inhibitor may act by preventing Smac release from the mitochondria, interfering with Smac binding to an IAP or by other mechanisms. The inhibitor may act directly or indirectly. Inhibitors include small molecules (organic molecules), peptides and polypeptides. In one embodiment, the inhibitors prevent apoptosis. Inhibitors should have a minimum of side effects and are preferably non-toxic.

In addition, enhancers of apoptotic activity are desirable in certain circumstances. At times, increasing apoptosis will have a therapeutic effect. For example, tumors or cells that mediate autoimmune diseases are appropriate cells for destruction. Enhancers may increase the rate or efficiency of caspase processing, increase transcription or translation, decrease proteolysis, or act through other mechanisms. As will be apparent to those skilled in the art, many of the guidelines presented above apply to the design of enhancers as well. Within the context of the present invention, Smac peptides, polypeptides, functional variants of each or functional equivalents of each can act as an enhancer. In one embodiment, the cytosolic form of Smac (Smac-S) can act as an enhancer of apoptosis in type II cells as disclosed in Example 5. In another embodiment, the Smac peptides, polypeptides, functional variants of each or functional equivalents of each can be used as promoters of caspase enzymatic activity at attainable concentrations to kill cancer cells that overexpress IAPs or as components in a chemotherapy regimen to sensitize cancers.

Screening assays for inhibitors and enhancers will vary according to the type of inhibitor or enhancer and the nature of the activity that is being affected. Assays may be performed in vitro or in vivo. In general, assays are designed to evaluate apoptotic pathway activation (e.g., caspase protein processing, caspase enzymatic activity, cell morphology changes, DNA laddering, and the like). In any of the assays, a statistically significant increase or decrease compared to a proper control is indicative of enhancement or inhibition. In one embodiment, the caspase utilized for the assays is selected from the group consisting of caspase-3, caspase-7 and caspase-9.

One in vitro assay can be performed by examining the effect of a candidate compound on the activation of an initiator caspase (e.g., caspase 9) or an effector caspase (e.g., caspases 3-7). Briefly, procaspase 9, an IAP, cytochrome c, dATP and a Smac peptide, polypeptide, functional variant or functional equivalent are provided. The processing of caspase-9 into two subunits can be assayed or alternatively caspase-9 enzymatic activity can be monitored by adding procaspase-3, procaspase-7, or other effector caspases and monitoring the activation of these caspases either directly via subunit formation or via substrate cleavage (e.g., acetyl DEVD-aminomethyl coumarin (amc), lamin, PRPP, PARP, and the like).

Further, to facilitate detection, typically the protein of interest may be in vitro translated and labeled during translation. This composition is incubated with a Smac peptide, polypeptide, functional variant or functional equivalent in the presence or absence of a candidate inhibitor or enhancer. Processing of caspase-9 into two subunits can be monitored as can processing/activation of a coincubated effector pro-caspase. Caspase processing is routinely monitored either by gel electrophoresis or indirectly by monitoring caspase substrate turnover. The two subunits and caspase substrate turnover may be readily detected by autoradiography after gel electrophoresis. One skilled in the art will recognize that other methods of labeling and detection may be used alternatively.

Moreover, any known enzymatic analysis can be used to follow the inhibitory or enhancing ability of a candidate compound with regard to the ability of Smac peptide of the present invention or variants thereof to promote the enzymatic activity of caspases. For example, one could express a Smac construct of interest in a cell line, be it bacterial, insect, mammalian or other, and purify the resulting polypeptide. The purified Smac peptide can then be used in a variety of assays to follow its ability to promote the enzymatic activity of effector caspases or apoptotic activity. Such methods of expressing and purifying recombinant proteins are known in the art and examples can be found in Sambrook et al., Molecular Cloning: A Laboratory Manual, Cold Spring Harbor Press, 1989 as well as in a number of other sources.

In vivo assays are typically performed in cells transfected either transiently or stably with an expression vector containing the Smac nucleic acid molecule such as those described herein. These cells are used to measure caspase processing, caspase substrate turnover, enzymatic activity of effector caspases or apoptosis in the presence or absence of a candidate compound. When assaying apoptosis, a variety of cell analyses may be used including, for example, dye staining and microscopy to examine nucleic acid fragmentation, porosity of the cells, and membrane blebbing.

A variety of methodologies exist that can be used to investigate the effect of a candidate compound. Such methodologies are those commonly used to analyze enzymatic reactions and include, for example, SDS-PAGE, spectroscopy, HPLC analysis, autoradiography, chemiluminescence, chromogenic reactions, and immunochemistry (e.g., blotting, precipitating, etc.).

2. Compositions and Methods of Modulating Apoptosis

Compositions comprising a Smac peptide, polypeptide, functional variant or functional equivalent as defined above are provided by the invention. Such compositions may be used to inhibit or promote apoptosis. In one embodiment, compositions comprising a nucleic acid molecule of the present invention, a peptide of the present invention or an antibody of the present invention; and a physiologically acceptable carrier.

These antibodies include, but are not limited to, polyclonal, monoclonal, single chain or humanized antibodies or antibody fragments. These compositions may comprise, for example, polyclonal antibodies that recognize one or more epitopes of Smac, particularly on the N-terminus. In one embodiment, an antibody recognizes an epitope that includes the amino acids AVPI (SEQ ID NO:13). Alternatively, they can comprise monoclonal antibodies that recognize specific epitopes of Smac. The antibodies of the composition may recognize native Smac and/or denatured Smac. These antibodies may be produced according to methods well known in the art, as described above.

Examples of polynucleotide compositions include mammalian expression vectors, sense RNAs, ribozymes, and antisense RNA. Expression vectors and sense RNA molecules are designed to express Smac, while ribozymes and antisense RNA constructs are designed to reduce the levels of the Smac expressed.

The compositions may also contain a physiologically acceptable carrier. The term “physiologically acceptable carrier” refers to a carrier for administration of a first component of the composition which is selected from antibodies, peptides or nucleic acids. Suitable carriers and physiologically acceptable salts are well known to those of ordinary skill in the art. A thorough discussion of acceptable carriers is available in Remington's Pharmaceutical Sciences, Mack Publishing Co., New Jersey, 1991).

Appropriate dosage amounts balancing toxicity and efficacy will be determined during any clinic testing pursued, but a typical dosage will be from about 0.001 mg/kg to 50 mg/kg or 0.05 mg/kg to about 10 mg/kg of the polynucleotide, peptide or antibody. If used in gene therapies such dosages will depend on the vector utilized and will be determined during any clinic testing pursued

The compositions of the invention can be (1) administered directly to the subject; (2) delivered ex vivo to cells derived from the subject; or (3) delivered in vitro. Direct delivery will generally be accomplished by injection. Alternatively, compositions can also be delivered via oral or pulmonary administration, suppositories, transdermally, or by gene guns, for example. Dosage treatment may be a single dose or multiple doses.

Methods of ex vivo delivery and reimplantation of transformed cells into a subject are known in the art. Generally, delivery of nucleic acids for both ex vivo and in vitro applications can be accomplished by, for example, dextran-mediated transfection, calcium phosphate precipitation transfection, viral infection, polybrene mediated transfection, protoplast fusion, electroporation, encapsulation of polynucleotides in liposomes, and direct microinjection of the DNA into nuclei, all well known in the art.

Gene therapy vectors comprising Smac nucleic acid sequences, or complements or variants thereof, are within the scope of the invention. These vectors may be used to regulate Smac mRNA and peptide or polypeptide expression in target cells. In some instances, it may be advantageous to increase the amount of Smac nucleic acids or Smac that are expressed. In other cases, gene therapy vectors may be used to decrease functional Smac levels. Gene therapy vectors may comprise any Smac nucleic acid of the current invention, including fragments, variants, antisense, ribozymes, and mutants. Expression of Smac nucleic acids may be controlled by endogenous mammalian or heterologous promoters and may be either constitutive or regulated. Nucleic acids used according to the invention may be stably integrated into the genome of the cell or may be maintained in the cell as separate episomal segments of DNA.

Smac nucleic acid molecules may be delivered by any method of gene delivery available in the art. Gene delivery vehicle may be of viral or non-viral origin (see generally Jolly, Cancer Gene Therapy 1:51-64, 1994; Kimura, Human Gene Therapy 5:845-852, 1994; Connelly, Human Gene Therapy 1:185-193, 1995; and Kaplitt, Nature Genetics 6:148-153, 1994). The present invention can employ recombinant retroviruses which are constructed to carry or express a Smac nucleic acid molecule. Methods of producing recombinant retroviral virions suitable for gene therapy have been extensively described (see, e.g., Mann et al. Cell 33:153-159, 1983; Nikolas and Rubenstein, Vectors: A survey of molecular cloning vectors and their uses, Rodriquez and Denhardt (eds.), Stoneham:Butterworth, 494-513, 1988).

The present invention also employs viruses such as alphavirus-based vectors, adenovirus, and parvovirus that can function as gene delivery vehicles. Examples of vectors utilized by the invention include intact adenovirus, replication-defective adenovirus vectors requiring a helper plasmid or virus, and adenovirus vectors with their native tropism modified or ablated such as adenoviral vectors containing a targeting ligand. Other examples include adeno-associated virus based vectors and lentivirus vectors.

Packaging cell lines suitable for use with the above-described viral and retroviral vector constructs may be readily prepared and used to create producer cell lines (also termed vector cell lines) for the production of recombinant vector particles.

Examples of non-viral methods of gene delivery vehicles and methods which may be employed according to the invention include liposomes (see, e.g., Wang et al. PNAS 84:7851-7855, 1987), polycationic condensed DNA (see, e.g., Curiel, Hum. Gene Ther. 3:147-154, 1992); ligand linked DNA (see, e.g., Wu, J. Biol. Chem. 264:16985-16987, 1989); deposition of photopolymerized hydrogel materials; hand-held gene transfer particle guns, as described in U.S. Pat. No. 5,149,655; ionizing radiation as described in U.S. Pat. No. 5,206,152 and WO 92/11033; and nucleic charge neutralization or fusion with cell membranes. Additional approaches are described in Philip, Mol. Cell Biol. 14:2411-2418, 1994 and in Woffendin, Proc. Natl. Acad. Sci. 91:1581-1585, 1994. Conjugates comprising a receptor-binding internalized ligand capable of delivering nucleic acids may also be used according to the present invention. Conjugate-based preparations and methods of use thereof are described in WO 96/36362 which is hereby incorporated by reference in its entirety. Other non-viral delivery methods include, but are not limited to, mechanical delivery systems such as the approach described in Woffendin et al., Proc. Natl. Acad. Sci. USA 91(24):11581-11585, 1994 and naked DNA protocols. Exemplary naked DNA introduction methods are described in WO 90/11092 and U.S. Pat. No. 5,580,859.

In other embodiments, methods of the invention utilize bacteriophage delivery systems capable of transfecting eukaryotic cells. Bacteriophage-mediated gene transfer systems are described in WO 99/10014 which is incorporated in its entirety. Phage delivery vehicles may express a targeting ligand on their surface which facilitates receptor-mediated gene delivery.

In addition, compositions and methods of modulating apoptosis using small molecule agonists or antagonists or heterologous polypeptides which bind the Smac are included within the scope of the current invention.

### EXAMPLES

The following experimental examples are offered by way of illustration, not limitation.

### Example 1

### Identification of a Cytosolic Isoform of Smac

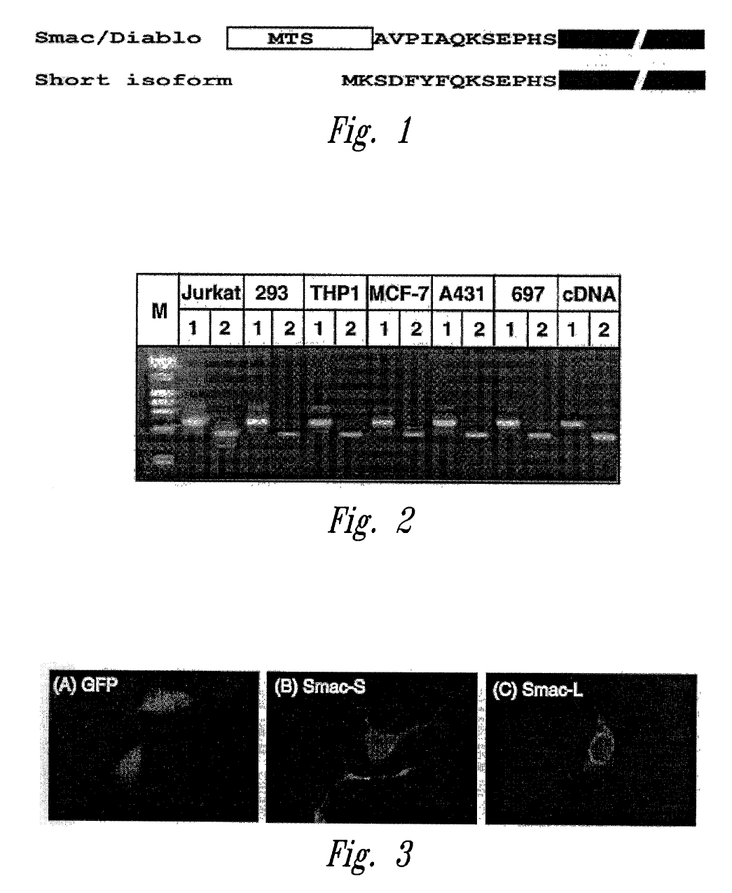

This example discloses the identification of a cytosolic isoform of Smac. The Smac precursor contains a 55-residue mitochondrial targeting sequence (MTS) at its N-terminus, which is cleaved in the mitochondria to generate the mature Smac. Genomic analysis of the Smac gene on chromosome 12q (Genbank accession # AC048338) revealed that the MTS is encoded by the first two exons. It was discovered that the exons encoding the MTS are spliced out in two human EST clones (AA156765, and AA305624) and one full length clone (AK001399) in the database. The three clones contain an open reading frame, which generates a short Smac isoform (Smac-S) that begins with the MKSDFYF sequence (SEQ ID NO:3), which replaces the MTS and residues 56-60 (AVPIA-SEQ ID NO:4) of the long Smac isoform (Smac-L) (see FIG. 1). Smac-S and Smac-L are 100% identical after reside 60.

The entire open reading frames of human Smac-L and Smac-S cDNAs were cloned from Jurkat mRNA by RT-PCR using complementary PCR adaptor primers spanning the initiation and stop codons of these cDNAs. The PCR primers were designed based on the sequence of mouse DIABLO and human Genbank clones AW16150 and AK001399. All Smac constructs were cloned in pET 28(a) in the NcoI/XhoI site with the C-terminal tagged with a His6-tag. The His6-tagged Smac was purified using a Talon resin by standard affinity-purification procedures as described in Srinivasula et al., Mol. Cell. 1:949-957, 1998.

The Smac constructs were transfected into different cell lines, Jurkat, 293, THP1, MCF7, A431 and 697. The alternatively spliced Smac-S mRNA was expressed in all cell lines tested by RT-PCR using isoform specific primers, although at a lower level than that of Smac-L (see FIG. 2).

Smac constructs were also constructed which encode GFP-fusion proteins of the two isoforms. The constructs were transiently transfected of into MCF-7 cells. Twenty-four hours after transfection, the cells were visualized by confocal microscopy and photographed. As shown in FIG. 3, the photographs revealed that Smac-S is targeted to the cytosol, whereas Smac-L is targeted to the mitochondria.

### Example 2

### Smac Functions at the Initiation and Effector Steps of the Caspase Cascade

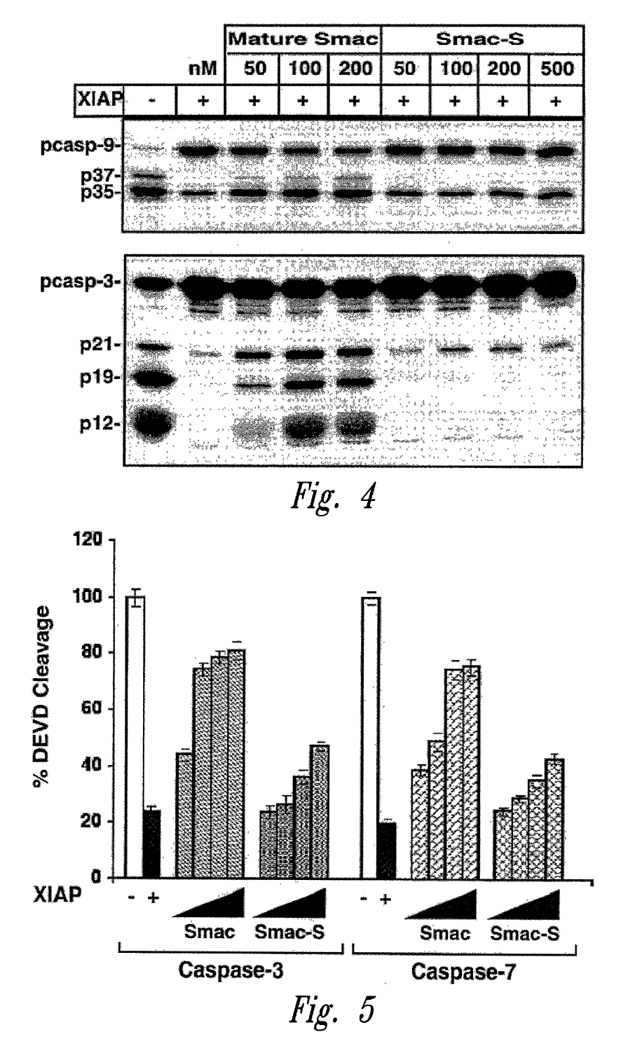

The interaction between the Smac isoforms and IAPs was examined in this example. Smac has been shown to enhance cytochrome c-dependent activation of caspase-3 by neutralizing the inhibitory effect of IAPs in S100 extracts. To compare the activity of the two isoforms, Smac-S and Smac-L without its MTS (mature Smac) were expressed in bacteria, purified and examined for their ability to enhance cytochrome c-dependent activation of caspase-9 and caspase-3 in XIAP-containing S100 extracts. 293T S100 extracts were mixed with purified XIAP (20 nM) and then stimulated with cytochrome c plus dATP in the presence of varying amounts of purified mature Smac or Smac-S. For mature Smac, the amounts used were 50, 100 and 200 nM and for Smac-S, they were 50, 100, 200 and 500 nM. S100 extracts without XIAP were used as controls. The reactions were carried out in the presence of ³⁵S-labeled procaspase-9 or procaspase-3. After a one-hour incubation, the samples were analyzed by SDS-PAGE and autoradiography. As shown in FIG. 4, mature Smac was able to relieve the inhibitory effect of XIAP and enhance caspase-9 (upper panel) and caspase-3 (lower panel) activation in a dose-dependent manner. Smac-S had a greatly reduced activity compared to mature Smac (see FIG. 4). Since the only difference between the two proteins is the substitution of the N-terminal AVPIA sequence (SEQ ID NO:4) in mature Smac with MKSDFYF (SEQ ID NO:3) in Smac-S, this indicated that the first 5 N-terminal residues of mature Smac are critical for its activation of caspase-9.

XIAP has been shown to inhibit the enzymatic activity of the effector caspases, caspase-3 and caspase-7. To determine whether Smac could also relieve the XIAP-inhibitory effect on the enzymatic activity of mature caspase-3 and -7, the effect of Smac and Smac-S on the activity of caspase-3 and -7 was tested in the presence of XIAP. Purified caspase-3 or caspase-7 were mixed with XIAP (20 nM in case of caspase-3, 5 nM in case of caspase-7) and the mixtures were then incubated with increasing amounts of purified mature Smac or Smac-S (100, 200, 500 or 1000 nM, respectively). The reactions were carried out in the presence of the peptide substrate acetyl DEVD-aminomethyl coumarin (DEVD-AMC) (50 μM) for 30 minutes. The release of AMC from the DEVD-AMC substrate was measured by luminescence spectrometry using a Perkin Elmer Luminescence spectrometer. The caspase activity in all the samples was plotted as a percentage of the activity of caspase-3 or caspase-7 in the absence of XIAP (100%). Smac was able to promote the enzymatic activity of both caspase-3 and caspase-7 (see FIG. 5). Interestingly, compared to its very low activity with caspase-9, Smac-S had ˜30% of the activity of wild type Smac with caspase-3 and -7 (see FIG. 5). This result indicates that Smac-S plays a role in regulating caspase-3 and -7 activity in vivo. Combined, these data indicate that Smac has a dual role in the caspase cascade, to promote activation of the initiator caspase-9 and to enhance the activity of the effector caspases.

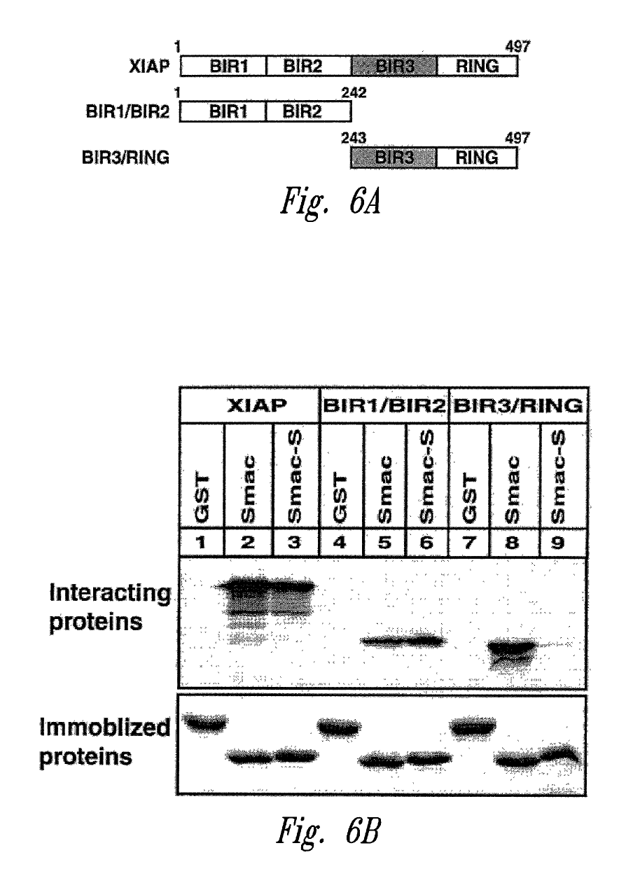

Since the ability of Smac to promote the enzymatic activity of caspases depends on its interaction with IAPs, it was determined whether the weak activity of Smac-S is due to altered interaction with XIAP. In vitro interaction assays were performed with ³⁵S-labeled full length XIAP, or isolated BIR domains of XIAP. As shown in FIG. 6A, XIAP is made up of four domains, the BIR1 domain followed by the BIR2 domain at the N-terminus and the BIR3 domain followed by RING domain at the C-terminus (Yang and Li, Cell Res. 10:169-77, 2000). It was previously determined that the BIR3 domain binds and inhibits caspase-9, while the BIR1/BIR2 domains are the domains that are important for caspase-3 and caspase-7 inhibition (Deveraux et al., Embo J. 18:5242-5251, 1999).

The in vitro interactions were performed by expressing mature Smac or Smac-S in bacteria with His6-tag and then immobilizing onto a Talon-affinity resin. The resin was incubated with in vitro translated ³⁵S-labeled XIAP, or BIR1/BIR2 or BIR3/RING domains of XIAP, washed at least 4 times and then analyzed by SDS-PAGE and autoradiography. A His6-tagged GST was used as a negative control. Consistent with its weak activity, Smac-S was not able to interact with the BIR3 domain (XIAP-BIR3/RING) of XIAP, but was still able to interact with the BIR1/BIR2 domains (XIAP-BIR1/2) of XIAP (see FIG. 6B). Smac-S was also able to interact with full length XIAP, although to a lesser extent than the mature Smac (see FIG. 6B).

### Example 3

### Smac's Caspase Promoting Activity Resides in its N-terminus

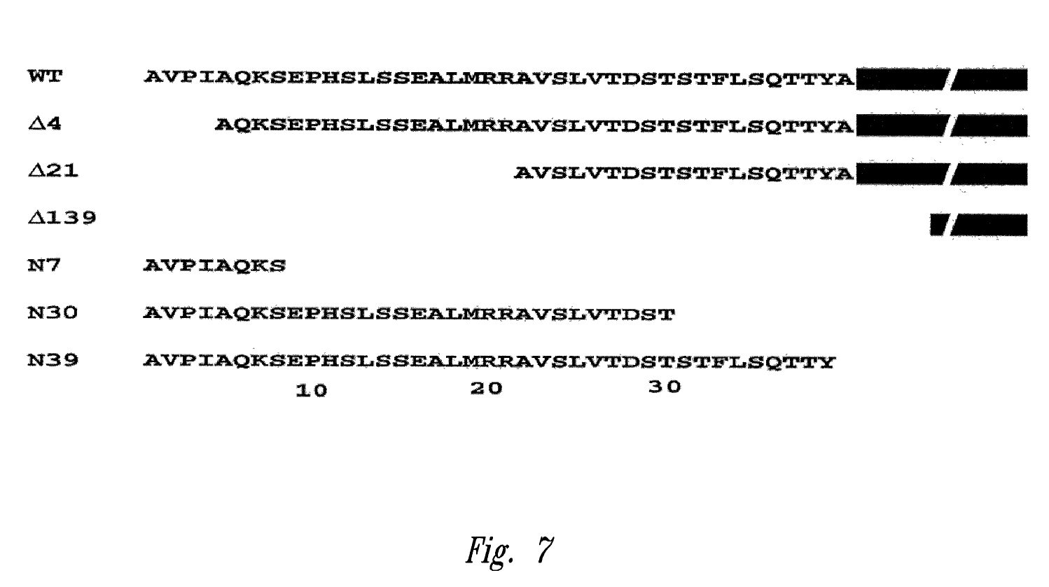

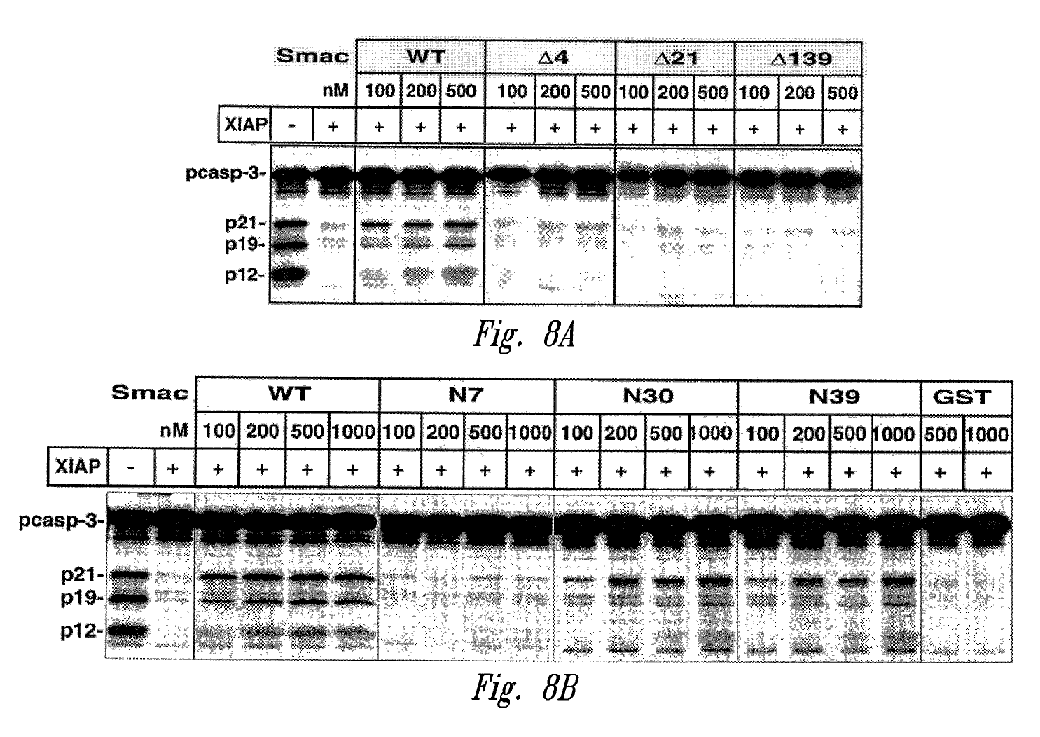

This example discloses that the caspase promoting activity resides in its N-terminus of Smac. A series of Smac N-terminal deletion mutants were generated with His6-tags and expressed in bacteria (see FIG. 7). The recombinant proteins were purified to homogeneity and assayed for their ability to promote cytochrome c-dependent caspase-3 activation in XIAP-containing S100 extracts as performed in Example 2. 100, 200 and 500 nM of purified mature Smac or mutants were tested in the assay. As shown in FIG. 8A, deletion of the first 4 residues of mature Smac (Δ4) dramatically reduced Smac activity to a level similar to that seen with Smac-S. Deletion of the first 21 residues (Δ21) further reduced Smac activity to undetectable levels. Other larger N-terminal deletions produced insoluble mutant proteins, which could not be used in this assay. However, one N-terminal deletion mutant lacking the first 139 residues (Δ139) was soluble but found to be completely inactive (see FIG. 8A).

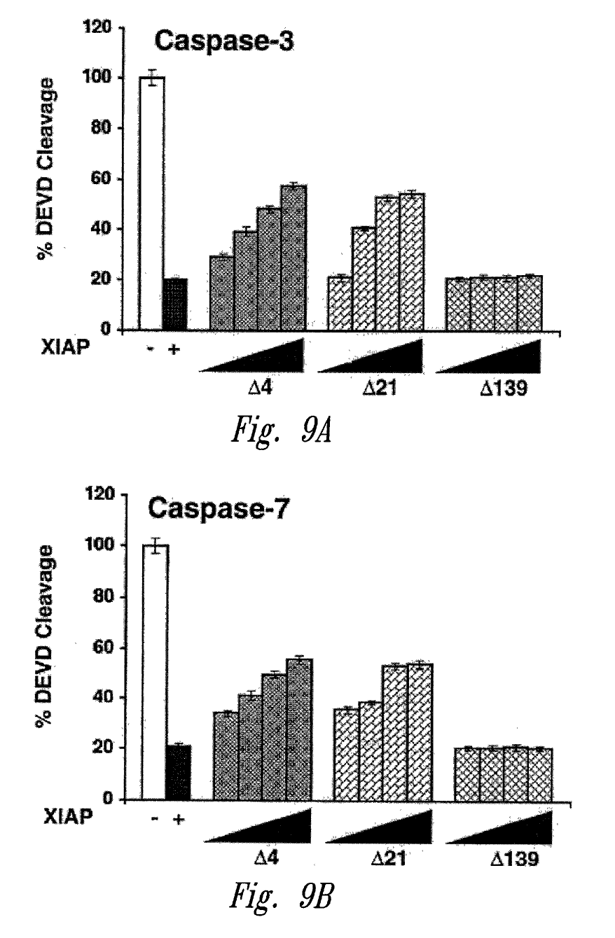

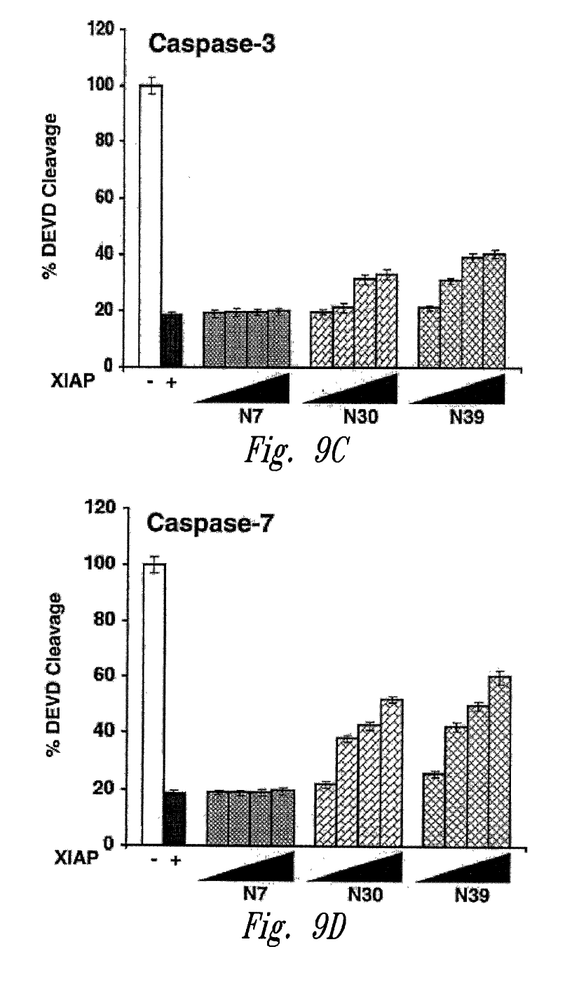

The N-terminal deletion mutants were then assayed for their ability to enhance caspase-3 and caspase-7 activity in the presence of XIAP. Like Smac-S, the N-terminal deletion mutants Δ4 and Δ21 had ˜30-40% of the activity of wild type Smac with caspase-3 and capase-7 (see FIGS. 9A and 9C). However, the N-terminal deletion mutant Δ139 was completely inactive in this assay (see FIGS. 9A and 9C).

The results with the N-terminal deletion mutants indicate that the N-terminus harbors the caspase-promoting activity of Smac. To confirm this, three C-terminal deletion mutants fused at their C-termini to GST were generated (see FIG. 7). These mutants, which contain the first 7, 30 or 39 N-terminal residues of mature Smac (N7, N30, N39, respectively-SEQ ID NO:6, SEQ ID NO:7 and SEQ ID NO:8, respectively), were expressed in bacteria and purified to homogeneity. Reactions were carried out with increasing amounts of purified mature Smac or mutants (100, 200, 500 or 1000 nM, respectively). As shown in FIG. 8B, N30 and N39 were able to promote caspase-3 activation in S100 extracts containing XIAP, although at a higher concentration than the wild type Smac. The smallest mutant, N7, was the least effective among the mutants (see FIGS. 8B).

Next the C-terminal mutants were tested for their effects on the enzymatic activity of caspase-3 and caspase-7. Caspase-3 and caspase-7 enzymatic assays were performed in a 20 μl volume at 37° C. The assay was performed as provided in Example 2. As shown in FIGS. 9B and 9D, N30 and N39 were able to relieve the XIAP inhibition of caspase-7 and caspase-3, although they were slightly more effective with the caspase-7 than caspase-3. However, within the range of concentrations used in this experiment, N7 had no detectable activity with caspase-3 and caspase-7 (see FIGS. 9B and 9D). Taken together, these results indicate that the caspase-promoting activity of Smac resides within an approximately 30 residue-long domain at its N-terminus.

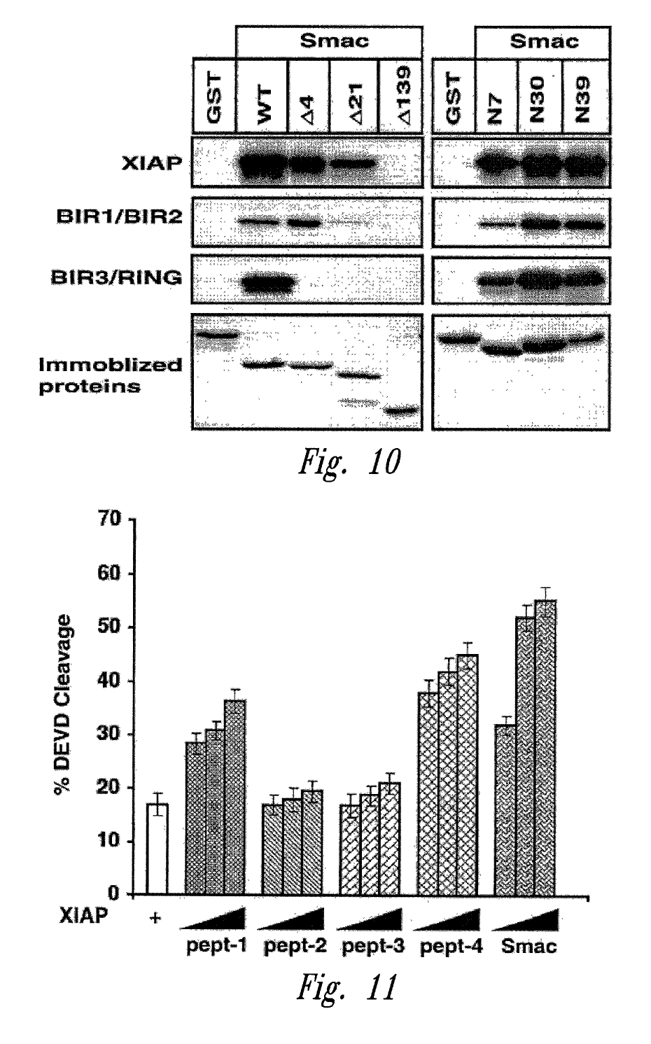

As performed in Example 2, the N-terminal and C-terminal mutants were tested for their ability to interact with XIAP in in vitro interaction assays performed with ³⁵S-labeled full length XIAP, or isolated BIR domains of XIAP. Like Smac-S, the N-terminal deletion mutants Δ4 and Δ21 were not able to interact with the BIR3 domain (XIAP-BIR3/RING) of XIAP, but were still able to interact with the BIR1/BIR2 domains (XIAP-BIR1/2) of XIAP, and to a slightly lesser extent with full length XIAP (see FIG. 10). Based on these observations, the first 4 residues of mature Smac appear essential for its ability to interact with the BIR3 domain of XIAP. Since the BIR3 domain of XIAP is the domain which binds and inhibits caspase-9, this could explain the very weak activity of Smac-S and the N-terminal deletion mutants Δ4 and Δ21 in the caspase-3 activation assay, which measures caspase-9 activity. Thus, deletion or substitution of the first 4 residues impairs the ability of Smac to interact with the BIR3 domain of XIAP and consequently its ability to promote caspase-3 activation by the caspase-9 apoptosome in the presence of XIAP. Nevertheless, since Smac-S and the N-terminal deletion mutants Δ4 and Δ21 can still interact with the BIR1/BIR2 domains of XIAP, which is more important for caspase-3 and -7 inhibition, they had better caspase promoting activity with caspase-3 and -7, compared to that with caspase-9 in the presence of XIAP.

Interestingly, all the three C-terminal deletion mutants (N7, N30 and N39) were able to interact with full length XIAP as well as the isolated BIR domains of XIAP (see FIG. 10). However, the interaction of N7 with XIAP and its isolated BIR domains was weaker than that observed with N30 and N39 (see FIG. 10).

### Example 4

### Smac N-terminal Peptides Promote Caspase Activation

This example discloses that the peptides from the N-terminus of Smac can promote caspase activation. Four peptides were chemically synthesized, based on the N-terminal sequences of mature Smac and Smac-S, and tested for their ability to promote cytochrome c-dependent activation of caspase-3 in S100 extracts containing XIAP. The four synthesized peptides were: peptide 1-AVPIAQK (the first 7 residues of mature Smac, Smac-N7, SEQ ID NO:6), peptide 2-MKSDFYFQK (Smac-S-N9, SEQ ID NO:9), peptide 3-TDSTSTFL (an internal Smac sequence, Smac15-35, SEQ ID NO:10) and peptide 4-AVPIAQKSEPHSLSSEALMRRAVSLVTDSTSTFLS (the first 35 residues of mature Smac, Smac-N35, SEQ ID NO:11). 293T S100 extracts were mixed with purified XIAP (20 nM) and then stimulated with cytochrome c plus dATP in the presence of increasing amounts of Smac (25, 100, 500 nM) or the purified N-terminal Smac or Smac-S peptides (25, 100, 500 μM). The reactions were carried out in the presence of DEVD-AMC as a substrate, and performed as provided in Example 2. As shown in FIG. 11, Smac-N7 and Smac-N35 were very effective in promoting caspase-3 activation in the XIAP containing extracts at 100-500 μM concentrations. Smac-N35 was noticeably better than Smac-N7 in promoting caspase-3 activation (see FIG. 11). Smac-S-N9 or Smac15-35 was almost completely inactive in this assay (see FIG. 11). These results indicate that short peptides derived from the N-terminus of mature Smac could be used as promoters of caspase enzymatic activity at attainable concentrations to kill cancer cells that overexpress IAPs.

### Example 5

### Expression of Cytosolic Smac in Type II Cancer Cells

This example discloses that the expression of a cytosolic Smac converts a type II cell cancer to a type I cancer cell. In type II cells, such as breast adenocarcinoma MCF-7 cells, death receptor-induced apoptosis can be blocked by expression of Bcl-2 or Bcl-xL. Whereas, type I cells, such as B lymphoblastoid cell line SKW6.4, are sensitive to death receptor-induced apoptosis even when Bcl-1 or Bcl-xL are expressed. One explanation for this difference is that in type II cells, direct activation of the effector caspases by caspase-8 is blocked at the level of the effector caspases by IAPs, such as XIAP. For example, the cleavage of BID by caspase-8 is required to release Smac to neutralize the IAPs and allow direct activation of the effector caspases by caspase-8 (see FIG. 14). Accordingly, by expressing a cytosolic form of Smac, the type II cells should be made sensitive to death receptor-induced apoptosis.

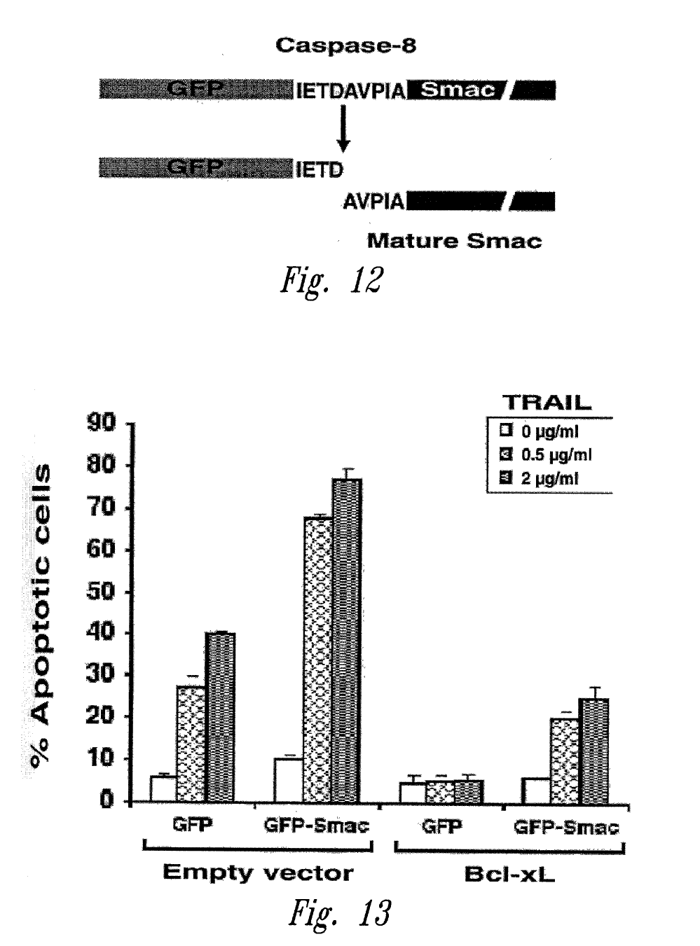

A mammalian GFP-Smac expression construct was constructed which allows the expression of a cytosolic GFP-Smac fusion protein that can be cleaved by caspase-8 to generate mature Smac. This was achieved by introduction of a caspase-8 cleavage site caspase-8 cleavage site, IETD, between a N-terminal GFP and a C-terminal mature Smac, i.e., IETD-AVPIA (SEQ ID NO:12) (see FIG. 12). Thus, upon stimulation of the death receptor by Trail, caspase-8 cleaves the fusion protein at this cleavage site and releases the cytosolic Smac (see FIG. 12).

MCF-7 cells (0.5×10⁵ cells/well) in 12 well plates were transfected with 0.5 μg of pEGFP-N1 reporter plasmid (Clontech) or GFP-Smac expression construct together with 0.5 μg of empty vector plasmids or plasmids encoding Bcl-xL using the LipofectAMINE™ method. Twenty-four hours after transfection cells were treated with TRAIL (0.5 or 2 μg/ml) for ten hours and then the normal (flat and attached) and apoptotic (round and detached) GFP-expressing cells were counted using fluorescence microscopy. The percentage of apoptotic cells in each experiment was expressed as the mean percentage of apoptotic cells as a fraction of the total number of GFP expression cells. Treatment of MCF7 cells with TRAIL, induced apoptosis in ˜28-40% of the cells. Overexpression of Bcl-xL inhibited TRAIL-induced apoptosis of MCF-7 cells confirming previous observations that these cells require the mitochondrial pathway for death receptor signaling. Interestingly, transfection of GFP-Smac into the Bcl-xL-expressing MCF7 cells bypassed the Bcl-xL inhibition and sensitized these cells to TRAIL-induced apoptosis to a level almost similar to that observed in the absence of Bcl-xL (˜22-30% apoptosis) (see FIG. 13). Moreover, transfection of GFP-Smac into MCF7 cells in the absence of overexpressed Bcl-xL, potentiated TRAIL-induced apoptosis and resulted in ˜65-80% cell death (see FIG. 13). The ability of GFP-Smac to potentiate TRAIL induced apoptosis in the absence of overexpressed Bcl-xL is consistent with the presence of an IAP block in these cells. This was confirmed by the finding that MCF-7 cells express high levels of XIAP. These results indicate that the inability of death receptor ligands to induce apoptosis in type II cells in the presence of overexpressed Bcl-xL is most likely attributed to inhibition of the effector caspases by IAPs. This inhibition can be only be bypassed by release of Smac from the mitochondria, a function that is performed by BID in type II cells after caspase-8 cleavage (see FIG. 14). Therefore, if Smac gene inactivation is not proven to be lethal in the future, it is expected that the phenotype of Smac-deficient mice would be similar to that of the BID-deficient mice with respect to sensitivity of normal hepatocytes to FAS-induced apoptosis. Since normal hepatocytes are type II cells, it is predicted that Smac gene inactivation should make these cells resistant to Fas-induced apoptosis.

In providing the forgoing description of the invention, citation has been made to several references that will aid in the understanding or practice thereof. All such references are incorporated by reference herein.

From the foregoing it will be appreciated that, although specific embodiments of the invention have been described herein for purposes of illustration, various modifications may be made without deviating from the spirit and scope of the invention. In addition, all references including patents, patent applications, and journal articles are incorporated herein in their entirety. Accordingly, the invention is not limited except as by the appended claims.

1. An isolated nucleic acid molecule comprising a polynucleotide having a sequence encoding a peptide or polypeptide of Smac having at least two contiguous amino acid residues derived from at least residues 56-139 of SEQ ID NO:1 and of which up to 184 contiguous amino acid residues can be derived from residues 56-239 of SEQ ID NO:1, a functional variant of each or a functional equivalent of each, each of which is capable of specifically binding to at least a portion of an Inhibitor of Apoptosis protein.

2. The isolated nucleic acid molecule of claim 1, wherein said portion is at least one BIR domain.

3. The isolated nucleic acid molecule of claim 2, wherein said BIR domain is BIR1.

4. The isolated nucleic acid molecule of claim 2, wherein said BIR domain is BIR2.

5. The isolated nucleic acid molecule of claim 2, wherein said BIR domain is BIR3.

6. The isolated nucleic acid molecule of claim 1, wherein said specific binding is to a full-length IAP

7. The isolated nucleic acid molecule of claim 1, wherein said peptide or polypeptide has an amino acid sequence of at least Ala-Val.

8. The isolated nucleic acid molecule of claim 1, wherein said peptide or polypeptide has an amino acid sequence of at least the sequence provided in SEQ ID NO:13.

9. An isolated nucleic acid molecule consisting essentially of a polynucleotide having a sequence encoding a peptide or polypeptide of Smac having at least two contiguous amino acid residues derived from at least residues 56-139 of SEQ ID NO:1 and of which up to 184 contiguous amino acid residues can be derived from residues 56-239 of SEQ ID NO:1, a functional variant of each or a functional equivalent of each, each of which is capable of specifically binding to at least a portion of an Inhibitor of Apoptosis protein.

10. The isolated nucleic acid molecule of claim 9, wherein said portion is at least one BIR domain.

11. The isolated nucleic acid molecule of claim 10, wherein said BIR domain is BIR1.

12. The isolated nucleic acid molecule of claim 10, wherein said BIR domain is BIR2.

13. The isolated nucleic acid molecule of claim 10, wherein said BIR domain is BIR3.

14. The isolated nucleic acid molecule of claim 9, wherein said specific binding is to a full-length IAP

15. The isolated nucleic acid molecule of claim 9, wherein said peptide or polypeptide has an amino acid sequence of at least Ala-Val.

16. The isolated nucleic acid molecule of claim 9, wherein said peptide or polypeptide has an amino acid sequence of at least the sequence provided in SEQ ID NO:13.

17. An isolated nucleic acid molecule consisting of a polynucleotide having a sequence encoding a peptide or polypeptide of Smac having at least two contiguous amino acid residues derived from at least residues 56-139 of SEQ ID NO:1 and of which up to 184 contiguous amino acid residues can be derived from residues 56-239 of SEQ ID NO:1, a functional variant of each or a functional equivalent of each, each of which is capable of specifically binding to at least a portion of an Inhibitor of Apoptosis protein.

18. The isolated nucleic acid molecule of claim 17, wherein said portion is at least one BIR domain.

19. The isolated nucleic acid molecule of claim 18, wherein said BIR domain is BIR1.

20. The isolated nucleic acid molecule of claim 18, wherein said BIR domain is BIR2.

21. The isolated nucleic acid molecule of claim 18, wherein said BIR domain is BIR3.

22. The isolated nucleic acid molecule of claim 17, wherein said specific binding is to a full-length IAP

23. The isolated nucleic acid molecule of claim 17, wherein said peptide or polypeptide has an amino acid sequence of at least Ala-Val.

24. The isolated nucleic acid molecule of claim 17, wherein said peptide or polypeptide has an amino acid sequence of at least the sequence provided in SEQ ID NO:13.

25. An expression vector comprising a nucleic acid molecule of claim 1 operatively linked to regulatory elements.

26. The expression vector of claim 25, wherein the regulatory elements include an inducible promoter.

27. A host cell transformed with an expression vector of claim 25.

28. An isolated peptide or polypeptide comprising an amino acid sequence having at least two contiguous amino acid residues derived from at least residues 56-139 of SEQ ID NO:1 and of which up to 184 contiguous amino acid residues can be derived from residues 56-239 of SEQ ID NO:1, a functional variant of each or a functional equivalent of each, each of which is capable of specifically binding to at least a portion of an Inhibitor of Apoptosis protein.

29. The isolated peptide or polypeptide of claim 28, wherein said portion is at least one BIR domain.

30. The isolated peptide or polypeptide of claim 29, wherein said BIR domain is BIR1.

31. The isolated peptide or polypeptide of claim 29, wherein said BIR domain is BIR2.

32. The isolated peptide or polypeptide of claim 29, wherein said BIR domain is BIR3.

33. The isolated peptide or polypeptide of claim 28, wherein said specific binding is to a full-length IAP

34. The isolated peptide or polypeptide of claim 28, wherein said peptide or polypeptide has an amino acid sequence of at least Ala-Val.

35. The isolated peptide or polypeptide of claim 28, wherein said peptide or polypeptide has an amino acid sequence of at least the sequence provided in SEQ ID NO:13.

36. An isolated Smac peptide or polypeptide consisting essentially of an amino acid sequence having at least two contiguous amino acid residues derived from at least residues 56-139 of SEQ ID NO:1 and of which up to 184 contiguous amino acid residues can be derived from residues 56-239 of SEQ ID NO:1, a functional variant of each or a functional equivalent of each, each of which is capable of specifically binding to at least a portion of an Inhibitor of Apoptosis protein.

37. The isolated peptide or polypeptide of claim 36, wherein said portion is at least one BIR domain.

38. The isolated peptide or polypeptide of claim 37, wherein said BIR domain is BIR1.

39. The isolated peptide or polypeptide of claim 37, wherein said BIR domain is BIR2.

40. The isolated peptide or polypeptide of claim 37, wherein said BIR domain is BIR3.

41. The isolated peptide or polypeptide of claim 36, wherein said specific binding is to a full-length IAP

42. The isolated peptide or polypeptide of claim 36, wherein said peptide or polypeptide has an amino acid sequence of at least Ala-Val.

43. The isolated peptide or polypeptide of claim 36, wherein said peptide or polypeptide has an amino acid sequence of at least the sequence provided in SEQ ID NO:13.

44. An isolated Smac peptide or polypeptide consisting of an amino acid sequence having at least two contiguous amino acid residues derived from at least residues 56-139 of SEQ ID NO:1 and of which up to 184 contiguous amino acid residues can be derived from residues 56-239 of SEQ ID NO:1, a functional variant of each or a functional equivalent of each, each of which is capable of specifically binding to at least a portion of an Inhibitor of Apoptosis protein.

45. The isolated peptide or polypeptide of claim 44, wherein said portion is at least one BIR domain.

46. The isolated peptide or polypeptide of claim 45, wherein said BIR domain is BIR1.

47. The isolated peptide or polypeptide of claim 45, wherein said BIR domain is BIR2.

48. The isolated peptide or polypeptide of claim 45, wherein said BIR domain is BIR3.

49. The isolated peptide or polypeptide of claim 44, wherein said specific binding is to a full-length IAP

50. The isolated peptide or polypeptide of claim 44, wherein said peptide or polypeptide has an amino acid sequence of at least Ala-Val.

51. The isolated peptide or polypeptide of claim 44, wherein said peptide or polypeptide has an amino acid sequence of at least the sequence provided in SEQ ID NO:13.

52. A method for inducing apoptosis in a cell, comprising contacting the cell with at least one component selected from the group consisting of:
(a) a peptide or polypeptide of claim 28 and
(b) a nucleic acid molecule one of claim 1,
under conditions and for a time sufficient to permit the induction of apoptosis in the cell.

53. A method of stimulating apoptosis in a neoplastic or tumor cell, comprising contacting the cell with at least one component selected from the group consisting of:
(a) a peptide or polypeptide of claim 28 and
(b) a nucleic acid molecule of claim 1,
under conditions and for a time sufficient to permit the induction of apoptosis in the cell.

54. The method of claim 53, wherein said cell overexpresses an inhibitor of a caspase.

55. The method of claim 54, wherein the inhibitor inhibits activation or activity of a caspase selected from the group consisting caspase-3, caspase-7 and caspase-9.

56. The method of claim 55, wherein the inhibitor is at least a portion of an Inhibitor of Apoptosis protein.

57. A method of identifying an inhibitor or enhancer of a caspase-mediated apoptosis comprising:
(a) contacting a cell transformed or transfected with a vector expressing the peptide or polypeptide of claim 28 with a candidate inhibitor or candidate enhancer; and
(b) detecting cell viability,
wherein an increase in cell viability indicates the presence of an inhibitor and a decrease in cell viability indicates the presence of an enhancer.

58. A method of identifying an inhibitor or enhancer of a caspase-mediated apoptosis comprising:
(a) contacting a cell transformed or transfected with a vector expressing the peptide or polypeptide of claim 28 with a candidate inhibitor or candidate enhancer; and
(b) detecting the presence of large and small caspase subunits, and therefrom determining the level of caspase processing activity,
wherein a decrease in processing indicates the presence of an inhibitor and an increase in processing indicates the presence of an enhancer.

59. The method of claim 58, wherein the caspase detected is selected from the group consisting of caspase-3, caspase-7 and caspase-9.

60. A method for identifying a compound that inhibits apoptosis comprising:
(a) separately contacting a plurality of cell populations expressing a cytosolic Smac and an inhibitor of BID with a compound to be tested for apoptotic inhibiting activity;
(b) incubating said cell populations with a direct stimulus of the cell death pathway; and
(c) measuring the specific apoptotic activity of the cell populations, wherein inhibition of the specific apoptotic activity is indicative that said compound is an inhibitor of apoptosis.

61. The method of claim 60, wherein said direct stimulus of the cell death pathway is selected from the group consisting of Fas ligand, anti-Fas antibody and staurosporine UV and gamma irradiation.

62. The method of claim 60, wherein (c) further comprises lysing said cells and determining caspase activity in said lysate.

63. The method of claim 60, wherein said compound exhibits caspase inhibitory activity.

64. The method of claim 60, wherein said compound inhibits apoptosis by promoting the activity of a cell survival polypeptide.

65. The method of claim 60, wherein said compound exhibits cell death polypeptide inhibitory activity.

66. A method for identifying a compound that inhibits Smac binding to a Smac-binding molecule, comprising:
(a) contacting a candidate compound with a Smac peptide in the presence of a Smac-binding molecule; and
(b) detecting displacement or inhibition of binding of said Smac-binding molecule from said Smac peptide.

67. The method of claim 66, wherein the Smac-binding molecule is at least a portion of an IAP.

68. The method of claim 67, wherein said portion is at least one BIR domain.

69. The method of claim 68, wherein said BIR domain is BIR1.

70. The method of claim 68, wherein said BIR domain is BIR2.

71. The method of claim 68, wherein said BIR domain is BIR3

72. The method of claim 67, wherein the Smac-binding molecule is a full length IAP.

73. A method for identifying a compound that inhibits Smac from being to a Smac-binding molecule, comprising:
(a) contacting a candidate compound with a Smac peptide in the presence of a Smac-binding molecule; and
(b) performing a functional assay that confirms displacement of said Smac-binding molecule from said Smac peptide.

74. The method of claim 73, wherein the functional assay detects the presence of large and small caspase subunits, and therefrom determining the level of caspase processing activity, wherein a decrease in processing confirms displacement.

75. The method of claim 74, wherein the caspase detected is selected from the group consisting of caspase-3, caspase-7 and caspase-9.

76. The method of claim 75, wherein the functional assay detects the presence of a substrate cleavage product produced by a caspase cleavage of a substrate.

77. The method of claim 76, wherein said substrate is acetyl DEVD-aminomethyl coumarin.

78. An antibody that specifically binds to a peptide or polypeptide of claim 28.

79. An antibody that specifically binds to an epitope located on the N-terminus of Smac.

80. The antibody of claim 79, wherein said antibody inhibits the binding of Smac to at least a portion of an IPA.

81. The antibody of claim 80, wherein said portion is at least one BIR domain.

82. The antibody of claim 81, wherein said BIR domain is BIR1.

83. The antibody of claim 81, wherein said BIR domain is BIR2.

84. The antibody of claim 81, wherein said BIR domain is BIR3.

85. The antibody of claim 80, wherein said antibody inhibits the binding to a full-length IAP.

86. The antibody of claim 78, wherein said antibody binds to an epitope that includes the amino acid sequence provided in SEQ ID NO:13.

87. A composition comprising a nucleic acid molecule of the claim 1, and a physiologically acceptable carrier.

88. A composition comprising a peptide of claim 28, and a physiologically acceptable carrier.

89. A composition comprising an antibody of claim 78, and a physiologically acceptable carrier.

90. A composition comprising an antibody of claim 79, and a physiologically acceptable carrier.

91. An isolated nucleic acid molecule comprising a polynucleotide having a sequence encoding a cytosolic isoform of Smac.

92. An isolated nucleic acid molecule consisting essentially of a polynucleotide having a sequence encoding a cytosolic isoform Smac.

93. An isolated nucleic acid molecule consisting of a polynucleotide having a sequence encoding a cytosolic isoform of Smac.

94. An isolated polypeptide comprising an amino acid sequence for a cytosolic isoform of Smac.

95. An isolated polypeptide consisting essentially of an amino acid sequence for a cytosolic isoform of Smac.

96. An isolated polypeptide consisting of an amino acid sequence for a cytosolic isoform.


SEQUENCE LISTING (18 sequences)

SEQ ID NO: 1 - DNA, 1358 bp, Homo sapiens
  CDS (20)...(739)
ggcgtccgcgcgctgcacaatggcggctctgaagagttggctgtcgcgcagcgtaacttcattcttcaggtacagacagtgtttgtgtgttcctgttgtggctaactttaagaagcggtgtttctcagaattgataagaccatggcacaaaactgtgacgattggctttggagtaaccctgtgtgcggttcctattgcacagaaatcagagcctcattcccttagtagtgaagcattgatgaggagagcagtgtctttggtaacagatagcacctctacctttctctctcagaccacatatgcgttgattgaagctattactgaatatactaaggctgtttataccttaacttctctttaccgacaatatacaagtttacttgggaaaatgaattcagaggaggaagatgaagtgtggcaggtgatcataggagccagagctgagatgacttcaaaacaccaagagtacttgaagctggaaaccacttggatgactgcagttggtctttcagagatggcagcagaagctgcatatcaaactggcgcagatcaggcctctataaccgccaggaatcacattcagctggtgaaactgcaggtggaagaggtgcaccagctctcccggaaagcagaaaccaagctggcagaagcacagatagaagagctccgtcagaaaacacaggaggaaggggaggagcgggctgagtcggagcaggaggcctacctgcgtgaggattgagggcctgagcacactgccctgtctccccactcagtggggaaagcaggggcagatgccaccctgcccagggttggcatgactgtctgtgcaccgagaagaggcggcaggtcctgccctggccaatcaggcgagacgcctttgtgagctgtgagtgcctcctgtggtctcaggcttgcgctggacctggttcttagcccttgggcactgcaccctgtttaacatttcaccccactctgtacagctgctcttacccattttttttacctcacacccaaagcattttgcctacctgggtcagagagaggagtcctttttgtcatgcccttaagttcagcaactgtttaacctgttttcagtcttatttacgtcgtcaaaaatgatttagtacttgttccctctgttgggatgccagttgtggcagggggaggggaacctgtccagtttgtacgatttctttgtatgtatttctgatgtgttctctgatctgcccccactgtcctgtgaggacagctgaggccaaggagtgaaaaacctattactactaagagaaggggtgcagagtgtttacctggtgctctcaacaggacttaacatcaacaggacttaacacagaaaaaaa

SEQ ID NO: 2 - protein, 40 aa, Homo sapiens
AVPIAQKSEPHSLSSEALMRRAVSLVTDSTSTFLSQTTYA

SEQ ID NO: 3 - protein, 5 aa, Homo sapiens
  VARIANT (4)...(4): Xaa = Arg, Gln or Gly
QACXG

SEQ ID NO: 4 - protein, 7 aa, Homo sapiens
MKSDFYF

SEQ ID NO: 5 - protein, 5 aa, Homo sapiens
AVPIA

SEQ ID NO: 6 - protein, 7 aa, Homo sapiens
AVPIAQK

SEQ ID NO: 7 - protein, 30 aa, Homo sapiens
AVPIAQKSEPHSLSSEALMRRAVSLVTDST

SEQ ID NO: 8 - protein, 39 aa, Homo sapiens
AVPIAQKSEPHSLSSEALMRRAVSLVTDSTSTFLSQTTY

SEQ ID NO: 9 - protein, 9 aa, Homo sapiens
MKSDFYFQK

SEQ ID NO: 10 - protein, 8 aa, Homo sapiens
TDSTSTFL

SEQ ID NO: 11 - protein, 35 aa, Homo sapiens
AVPIAQKSEPHSLSSEALMRRAVSLVTDSTSTFLS

SEQ ID NO: 12 - protein, 9 aa, Homo sapiens
IETDAVPIA

SEQ ID NO: 13 - protein, 4 aa, Homo sapiens
AVPI

SEQ ID NO: 14 - protein, 4 aa, Homo sapiens
ATPF

SEQ ID NO: 15 - protein, 4 aa, Drosophila sp.
AVAF

SEQ ID NO: 16 - protein, 4 aa, Drosophila sp.
AVPF

SEQ ID NO: 17 - protein, 4 aa, Mus musculus
AVPY

SEQ ID NO: 18 - protein, 4 aa, Xenopus sp.
ATPV